(19) United States
(12) Patent Application Publication
(10) Pub. No.: US 2023/0335257 A1
(43) Pub. Date: Oct. 19, 2023

(54) ELECTRONIC APPARATUS FOR PROVIDING COACHING AND OPERATING METHOD THEREOF

(71) Applicant: SAMSUNG ELECTRONICS CO., LTD., Suwon-si (KR)

(72) Inventors: Jeongja KIM, Suwon-si (KR); Donghyun ROH, Suwon-si (KR); Kyungsub MIN, Suwon-si (KR); Jungwon LEE, Suwon-si (KR)

(21) Appl. No.: 18/213,148

(22) Filed: Jun. 22, 2023

Related U.S. Application Data
(63) Continuation of application No. PCT/KR2022/014819, filed on Sep. 30, 2022.

(30) Foreign Application Priority Data
Oct. 15, 2021 (KR) 10-2021-0137717

(51) Int. Cl. G16H 20/70; A61M 21/00

(52) U.S. Cl. CPC G16H 20/70; A61M 21/00; A61M 2021/0022; A61M 2021/0027; A61M 2021/0044

(57) ABSTRACT

Disclosed are an electronic device and an operation method thereof. An electronic device configured to detect occurrence of a coaching event, and determine a coaching message to be displayed based on the coaching event. The electronic device identifies at least one emotion tag related to the coaching message, and determines, based on user context information, a representative visual element from a visual element candidate group corresponding to the at least one emotion tag. The electronic device includes the representative visual element in the coaching message and displays the coaching message.

### CROSS-REFERENCE TO RELATED APPLICATION(S)

This application is a continuation application, claiming priority under § 365(c), of an International application No. PCT/KR2022/014819, filed on Sep. 30, 2022, which is based on and claims the benefit of a Korean patent application number 10-2021-0137717, filed on Oct. 15, 2021, in the Korean Intellectual Property Office, the disclosure of which is incorporated by reference herein in its entirety.

### TECHNICAL FIELD

The disclosure relates to an electronic device for providing coaching and an operation method thereof.

### BACKGROUND ART

Recently, the development of mobile communication technologies has generalized the usage of electronic devices (e.g., smartphones, mobile terminals, or wearable devices) having portability or mobility, and services or functions provided via such electronic devices have been diversified.

For example, such electronic devices may provide a healthcare service that continuously monitors user biometric data or user exercise data, sleep data, and/or diet data, and may manage health. The electronic device (e.g., a smartphone) may obtain user data from one or more sensors or external electronic devices (e.g., a wearable device such as a smart watch), and may analyze the condition of a user's health based on the obtained user data.

### SUMMARY

### Technical Problem

A coaching (or guidance) service via an electronic device may be provided in various forms, and text-based simple coaching that uses text may be a representative example.

In the case of the text-based simple coaching, a user needs to read and understand the content of text in detail, which may be uncomfortable. An electronic device provided in a small size (e.g., a smartphone, a mobile terminal, a wearable device) requires user interaction many times, and thus a user may feel more uncomfortable. For example, in order to identify the content of text provided during coaching, touching a screen or a gesture (e.g., a swipe) of swiping up or down may be repeatedly required.

In addition, in order to increase the effect of coaching, it is important to repeatedly show, to a user, text provided during coaching so as to induce habituation. In this instance, in the case that the content (e.g., the content of text) provided to the user scarcely changes, a user may easily get bored or may less concentrate on the content of coaching and thus, the effect of coaching may be decreased.

Various embodiments disclosed in the document provide an electronic device that implements coaching associated with a healthcare service in an intuitive and understandable manner, and an operation method thereof.

Various embodiments disclosed in the document provide an electronic device that increases the effect of coaching by properly expressing the content of coaching that a user needs, and increasing the level of empathy or interest to be appropriate for the user, and an operation method thereof.

Various embodiments disclosed in the document provide an electronic device that improves fun or unexpectedness of coaching when expressing the content of coaching that may be repeatedly provided or may be stodgy and boring, and an operation method thereof.

### Technical Solution

An electronic device according to various embodiments includes a memory, a display, a communication circuit, and at least one processor. The at least one processor is operatively connected to the memory, the display, and the communication circuit. The memory stores instructions that, when executed, cause the at least one processor to detect occurrence of a coaching event, to determine a coaching message to be displayed based on the coaching event, to identify at least one emotion tag related to the coaching message, to determine, based on user context information of a user, a representative visual element from a visual element candidate group corresponding to the at least one emotion tag, and to include the representative visual element in the coaching message and display the coaching message via the display.

An operation method of an electronic device according to various embodiments includes an operation of detecting occurrence of a coaching event, an operation of determining a coaching message to be displayed based on the coaching event, an operation of identifying at least one emotion tag related to the coaching message, an operation of determining, based on user context information of a user, a representative visual element from a visual element candidate group corresponding to the at least one emotion tag, and an operation of including the representative visual element in the coaching message and displaying the coaching message on a display of the electronic device.

### Advantageous Effects

According to various embodiments, coaching of a healthcare service can be implemented in an intuitive and understandable manner According to various embodiments, the effect of coaching can be increased by properly expressing the content of coaching that a user needs and improving the level of empathy or interest to be appropriate for the user.

According to various embodiments, the fun or unexpectedness of coaching can be improved when the content of coaching that may be repeatedly provided, or may be stodgy and boring is expressed.

In addition, various effects directly or indirectly recognized from the disclosure can be provided.

### DESCRIPTION OF DRAWINGS

FIG. 1 is a block diagram of an electronic device in a network environment, according to various embodiments.

FIG. 2 is a block diagram of an electronic device according to an embodiment.

FIG. 3 is a block diagram illustrating the configuration of modules of an electronic device and an external electronic device according to an embodiment.

FIG. 4 is a flowchart illustrating an operation method of an electronic device according to an embodiment.

FIG. 5 is a flowchart illustrating part of the operation method of the electronic device of FIG. 4.

FIG. 6 is a diagram illustrating an example of the mapping relationship between visual elements and emotion tags according to an embodiment.

FIG. 7 is a diagram illustrating examples of user interfaces displayable in an electronic device according to an embodiment.

FIG. 8 is a diagram illustrating other examples of user interfaces displayed in an electronic device according to an embodiment.

FIG. 9 is a diagram illustrating an example to describe a coaching condition of an electronic device according to an embodiment.

FIG. 10 is a diagram illustrating an example of a scheme of setting a coaching message and an emotion tag using a design tool according to an embodiment.

FIG. 11 is a diagram illustrating an example of a scheme that registers a new visual element using a design tool according to an embodiment.

### MODE FOR INVENTION

Hereinafter, various embodiments will be described with reference to the attached drawings.

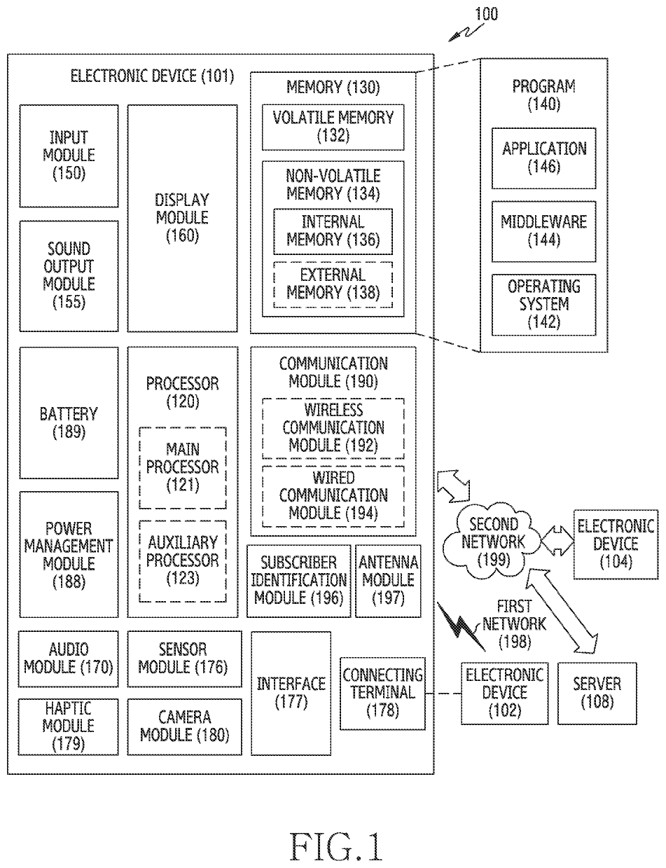

FIG. 1 is a block diagram illustrating an electronic device 101 in a network environment 100 according to various embodiments.

Referring to FIG. 1, the electronic device 101 in the network environment 100 may communicate with an electronic device 102 via a first network 198 (e.g., a short-range wireless communication network), or at least one of an electronic device 104 or a server 108 via a second network 199 (e.g., a long-range wireless communication network). According to an embodiment, the electronic device 101 may communicate with the electronic device 104 via the server 108. According to an embodiment, the electronic device 101 may include a processor 120, memory 130, an input module 150, a sound output module 155, a display module 160, an audio module 170, a sensor module 176, an interface 177, a connecting terminal 178, a haptic module 179, a camera module 180, a power management module 188, a battery 189, a communication module 190, a subscriber identification module (SIM) 196, or an antenna module 197. In some embodiments, at least one of the components (e.g., the connecting terminal 178) may be omitted from the electronic device 101, or one or more other components may be added in the electronic device 101. In some embodiments, some of the components (e.g., the sensor module 176, the camera module 180, or the antenna module 197) may be implemented as a single component (e.g., the display module 160).

The processor 120 may execute, for example, software (e.g., a program 140) to control at least one other component (e.g., a hardware or software component) of the electronic device 101 coupled with the processor 120, and may perform various data processing or computation. According to one embodiment, as at least part of the data processing or computation, the processor 120 may store a command or data received from another component (e.g., the sensor module 176 or the communication module 190) in volatile memory 132, process the command or the data stored in the volatile memory 132, and store resulting data in non-volatile memory 134. According to an embodiment, the processor 120 may include a main processor 121 (e.g., a central processing unit (CPU) or an application processor (AP)), or an auxiliary processor 123 (e.g., a graphics processing unit (GPU), a neural processing unit (NPU), an image signal processor (ISP), a sensor hub processor, or a communication processor (CP)) that is operable independently from, or in conjunction with, the main processor 121. For example, when the electronic device 101 includes the main processor 121 and the auxiliary processor 123, the auxiliary processor 123 may be adapted to consume less power than the main processor 121, or to be specific to a specified function. The auxiliary processor 123 may be implemented as separate from, or as part of the main processor 121.

The auxiliary processor 123 may control at least some of functions or states related to at least one component (e.g., the display module 160, the sensor module 176, or the communication module 190) among the components of the electronic device 101, instead of the main processor 121 while the main processor 121 is in an inactive (e.g., sleep) state, or together with the main processor 121 while the main processor 121 is in an active state (e.g., executing an application). According to an embodiment, the auxiliary processor 123 (e.g., an image signal processor or a communication processor) may be implemented as part of another component (e.g., the camera module 180 or the communication module 190) functionally related to the auxiliary processor 123. According to an embodiment, the auxiliary processor 123 (e.g., the neural processing unit) may include a hardware structure specified for artificial intelligence model processing. An artificial intelligence model may be generated by machine learning. Such learning may be performed, e.g., by the electronic device 101 where the artificial intelligence is performed or via a separate server (e.g., the server 108). Learning algorithms may include, but are not limited to, e.g., supervised learning, unsupervised learning, semi-supervised learning, or reinforcement learning. The artificial intelligence model may include a plurality of artificial neural network layers. The artificial neural network may be a deep neural network (DNN), a convolutional neural network (CNN), a recurrent neural network (RNN), a restricted boltzmann machine (RBM), a deep belief network (DBN), a bidirectional recurrent deep neural network (BRDNN), deep Q-network or a combination of two or more thereof but is not limited thereto. The artificial intelligence model may, additionally or alternatively, include a software structure other than the hardware structure.

The memory 130 may store various data used by at least one component (e.g., the processor 120 or the sensor module 176) of the electronic device 101. The various data may include, for example, software (e.g., the program 140) and input data or output data for a command related thereto. The memory 130 may include the volatile memory 132 or the non-volatile memory 134.

The program 140 may be stored in the memory 130 as software, and may include, for example, an operating system (OS) 142, middleware 144, or an application 146.

The input module 150 may receive a command or data to be used by another component (e.g., the processor 120) of the electronic device 101, from the outside (e.g., a user) of the electronic device 101. The input module 150 may include, for example, a microphone, a mouse, a keyboard, a key (e.g., a button), or a digital pen (e.g., a stylus pen).

The sound output module 155 may output sound signals to the outside of the electronic device 101. The sound output module 155 may include, for example, a speaker or a receiver. The speaker may be used for general purposes, such as playing multimedia or playing record. The receiver may be used for receiving incoming calls. According to an embodiment, the receiver may be implemented as separate from, or as part of the speaker.

The display module 160 may visually provide information to the outside (e.g., a user) of the electronic device 101. The display module 160 may include, for example, a display, a hologram device, or a projector and control circuitry to control a corresponding one of the display, hologram device, and projector. According to an embodiment, the display module 160 may include a touch sensor adapted to detect a touch, or a pressure sensor adapted to measure the intensity of force incurred by the touch.

The audio module 170 may convert a sound into an electrical signal and vice versa. According to an embodiment, the audio module 170 may obtain the sound via the input module 150, or output the sound via the sound output module 155 or a headphone of an external electronic device (e.g., an electronic device 102) directly (e.g., wiredly) or wirelessly coupled with the electronic device 101.

The sensor module 176 may detect an operational state (e.g., power or temperature) of the electronic device 101 or an environmental state (e.g., a state of a user) external to the electronic device 101, and then generate an electrical signal or data value corresponding to the detected state. According to an embodiment, the sensor module 176 may include, for example, a gesture sensor, a gyro sensor, an atmospheric pressure sensor, a magnetic sensor, an acceleration sensor, a grip sensor, a proximity sensor, a color sensor, an infrared (IR) sensor, a biometric sensor, a temperature sensor, a humidity sensor, or an illuminance sensor.

The interface 177 may support one or more specified protocols to be used for the electronic device 101 to be coupled with the external electronic device (e.g., the electronic device 102) directly (e.g., wiredly) or wirelessly. According to an embodiment, the interface 177 may include, for example, a high definition multimedia interface (HDMI), a universal serial bus (USB) interface, a secure digital (SD) card interface, or an audio interface.

A connecting terminal 178 may include a connector via which the electronic device 101 may be physically connected with the external electronic device (e.g., the electronic device 102). According to an embodiment, the connecting terminal 178 may include, for example, a HDMI connector, a USB connector, a SD card connector, or an audio connector (e.g., a headphone connector).

The haptic module 179 may convert an electrical signal into a mechanical stimulus (e.g., a vibration or a movement) or electrical stimulus which may be recognized by a user via his tactile sensation or kinesthetic sensation. According to an embodiment, the haptic module 179 may include, for example, a motor, a piezoelectric element, or an electric stimulator.

The camera module 180 may capture a still image or moving images. According to an embodiment, the camera module 180 may include one or more lenses, image sensors, image signal processors, or flashes.

The power management module 188 may manage power supplied to the electronic device 101. According to one embodiment, the power management module 188 may be implemented as at least part of, for example, a power management integrated circuit (PMIC).

The battery 189 may supply power to at least one component of the electronic device 101. According to an embodiment, the battery 189 may include, for example, a primary cell which is not rechargeable, a secondary cell which is rechargeable, or a fuel cell.

The communication module 190 may support establishing a direct (e.g., wired) communication channel or a wireless communication channel between the electronic device 101 and the external electronic device (e.g., the electronic device 102, the electronic device 104, or the server 108) and performing communication via the established communication channel. The communication module 190 may include one or more communication processors that are operable independently from the processor 120 (e.g., the application processor (AP)) and supports a direct (e.g., wired) communication or a wireless communication. According to an embodiment, the communication module 190 may include a wireless communication module 192 (e.g., a cellular communication module, a short-range wireless communication module, or a global navigation satellite system (GNSS) communication module) or a wired communication module 194 (e.g., a local area network (LAN) communication module or a power line communication (PLC) module). A corresponding one of these communication modules may communicate with the external electronic device via the first network 198 (e.g., a short-range communication network, such as Bluetooth™, wireless-fidelity (Wi-Fi) direct, or infrared data association (IrDA)) or the second network 199 (e.g., a long-range communication network, such as a legacy cellular network, a 5G network, a next-generation communication network, the Internet, or a computer network (e.g., LAN or wide area network (WAN)). These various types of communication modules may be implemented as a single component (e.g., a single chip), or may be implemented as multi components (e.g., multi chips) separate from each other. The wireless communication module 192 may identify and authenticate the electronic device 101 in a communication network, such as the first network 198 or the second network 199, using subscriber information (e.g., international mobile subscriber identity (IMSI)) stored in the subscriber identification module 196.

The wireless communication module 192 may support a 5G network, after a 4G network, and next-generation communication technology, e.g., new radio (NR) access technology. The NR access technology may support enhanced mobile broadband (eMBB), massive machine type communications (mMTC), or ultra-reliable and low-latency communications (URLLC). The wireless communication module 192 may support a high-frequency band (e.g., the mmWave band) to achieve, e.g., a high data transmission rate. The wireless communication module 192 may support various technologies for securing performance on a high-frequency band, such as, e.g., beamforming, massive multiple-input and multiple-output (massive MIMO), full dimensional MIMO (FD-MIMO), array antenna, analog beam-forming, or large scale antenna. The wireless communication module 192 may support various requirements specified in the electronic device 101, an external electronic device (e.g., the electronic device 104), or a network system (e.g., the second network 199). According to an embodiment, the wireless communication module 192 may support a peak data rate (e.g., 20 Gbps or more) for implementing eMBB, loss coverage (e.g., 164 dB or less) for implementing mMTC, or U-plane latency (e.g., 0.5 ms or less for each of downlink (DL) and uplink (UL), or a round trip of 1 ms or less) for implementing URLLC.

The antenna module 197 may transmit or receive a signal or power to or from the outside (e.g., the external electronic device) of the electronic device 101. According to an embodiment, the antenna module 197 may include an antenna including a radiating element composed of a conductive material or a conductive pattern formed in or on a substrate (e.g., a printed circuit board (PCB)). According to an embodiment, the antenna module 197 may include a plurality of antennas (e.g., array antennas). In such a case, at least one antenna appropriate for a communication scheme used in the communication network, such as the first network 198 or the second network 199, may be selected, for example, by the communication module 190 (e.g., the wireless communication module 192) from the plurality of antennas. The signal or the power may then be transmitted or received between the communication module 190 and the external electronic device via the selected at least one antenna. According to an embodiment, another component (e.g., a radio frequency integrated circuit (RFIC)) other than the radiating element may be additionally formed as part of the antenna module 197.

According to various embodiments, the antenna module 197 may form a mmWave antenna module. According to an embodiment, the mmWave antenna module may include a printed circuit board, a RFIC disposed on a first surface (e.g., the bottom surface) of the printed circuit board, or adjacent to the first surface and capable of supporting a designated high-frequency band (e.g., the mmWave band), and a plurality of antennas (e.g., array antennas) disposed on a second surface (e.g., the top or a side surface) of the printed circuit board, or adjacent to the second surface and capable of transmitting or receiving signals of the designated high-frequency band.

At least some of the above-described components may be coupled mutually and communicate signals (e.g., commands or data) therebetween via an inter-peripheral communication scheme (e.g., a bus, general purpose input and output (GPIO), serial peripheral interface (SPI), or mobile industry processor interface (MIPI)).

According to an embodiment, commands or data may be transmitted or received between the electronic device 101 and the external electronic device 104 via the server 108 coupled with the second network 199. Each of the electronic devices 102 or 104 may be a device of a same type as, or a different type, from the electronic device 101. According to an embodiment, all or some of operations to be executed at the electronic device 101 may be executed at one or more of the external electronic devices 102, 104, or 108. For example, if the electronic device 101 should perform a function or a service automatically, or in response to a request from a user or another device, the electronic device 101, instead of, or in addition to, executing the function or the service, may request the one or more external electronic devices to perform at least part of the function or the service. The one or more external electronic devices receiving the request may perform the at least part of the function or the service requested, or an additional function or an additional service related to the request, and transfer an outcome of the performing to the electronic device 101. The electronic device 101 may provide the outcome, with or without further processing of the outcome, as at least part of a reply to the request. To that end, a cloud computing, distributed computing, mobile edge computing (MEC), or client-server computing technology may be used, for example. The electronic device 101 may provide ultra low-latency services using, e.g., distributed computing or mobile edge computing. In another embodiment, the external electronic device 104 may include an internet-of-things (IoT) device. The server 108 may be an intelligent server using machine learning and/or a neural network. According to an embodiment, the external electronic device 104 or the server 108 may be included in the second network 199. The electronic device 101 may be applied to intelligent services (e.g., smart home, smart city, smart car, or healthcare) based on 5G communication technology or IoT-related technology.

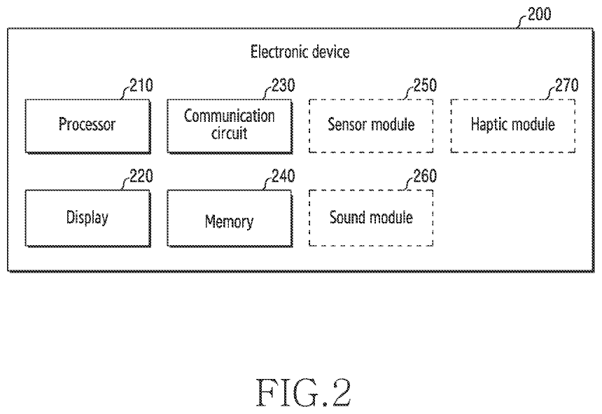

FIG. 2 is a block diagram of an electronic device according to an embodiment.

An electronic device 200 according to an embodiment may be to provide coaching (e.g., a coaching service, a coaching function). For example, the electronic device 200 may be embodied as one of the device types among a smartphone, a flexible smartphone, and a wearable device (e.g., a smart watch, smart glasses). Hereinafter, coaching according to various embodiments may be a function in which the electronic device 200 provides, to a user, a user interface (e.g., a graphic user interface (GUI), audio user interface (AUI)) including at least some of user health condition information, proposal (or recommendation) comment information associated with a user health condition, and/or activity performance information related to a user's health (e.g., exercise state measurement, diet records, weight reduction).

Referring to FIG. 2, an electronic device 200 according to an embodiment may include a processor 210, a display 220, and a communication circuit 230. The electronic device 200 may further include one or more among a memory 240, a sensor module 250, a sound module 260, and a haptic module 270. The electronic device 200 may omit at least one of the component elements or may additionally include another component element (e.g., at least some of the component elements of FIG. 1).

The component elements included in the electronic device 200 may be connected electrically and/or operatively and may exchange signals (e.g., commands or data) therebetween.

In FIG. 2, the component elements of the electronic device 200 may correspond to the component elements of the electronic device 101 of FIG. 1. For example, the processor 210 may correspond to the processor (one of the processors 120, 121, or 123) of FIG. 1. The display 220 may include the display module 160 of FIG. 1, or may correspond to the display module 160. The communication circuit 230 may include the communication module 190 of FIG. 1 The memory 240 may include at least a part of the memory 130 of FIG. 1. The sensor module 250 may correspond to the sensor module 176 of FIG. 1 or may include a part thereof. The sound module 260 may include at least one of the sound output module 155 and the audio module 170 of FIG. 1. The haptic module 270 may correspond to the haptic module 179 of FIG. 1.

According to an embodiment, the processor 210 may perform and/or control various functions supported in the electronic device 200. The processor 210 may control at least some of the display 220, the communication circuit 230, the memory 240, the sensor module 250, the sound module 260, and the haptic module 270. The processor 210 may perform code written in a programing language stored in the memory 240 of the electronic device 200, so as to perform an application and to control various pieces of hardware. For example, the processor 210 may perform an application for a healthcare service and/or a coaching service (e.g., a health application, an excise application, a fitness application, a sleep application, a diet management application), so as to provide a coaching function using the application. The application performed in the electronic device 200 may operate independently or may operate by interoperating with an external electronic device (e.g., the server 108, the electronic device 102, or the electronic device 104 of FIG. 1).

According to an embodiment, the processor 210 may include at least one processor. For example, the processor 210 may include a main processor (e.g., the main processor 121 of FIG. 1) and a sub-processor (e.g., the sub-processor 123 of FIG. 1). The main processor may be an application processor. The sub-processor may be a processor (e.g., a sensor hub-processor, a communication processor) configured to operate with power lower than the main processor, or to be specific to a designated function. The sub-processor may control the sensor module 250. The sub-processor may receive data from the sensor module 250, may process the data, and may transmit the processed data to the main processor. For example, even in the case that the main processor 121 is in a sleep state (or an idle state) since a user does not use the electronic device 200 for at least a predetermined period of time (e.g., 30 seconds), the sensor hub processor does not enter into the sleep state, and may process data collected via the sensor module 250 so as to improve the continuity and/or reliability of the data.

When instructions stored in the memory 240 are executed, the processor 210 may perform operations.

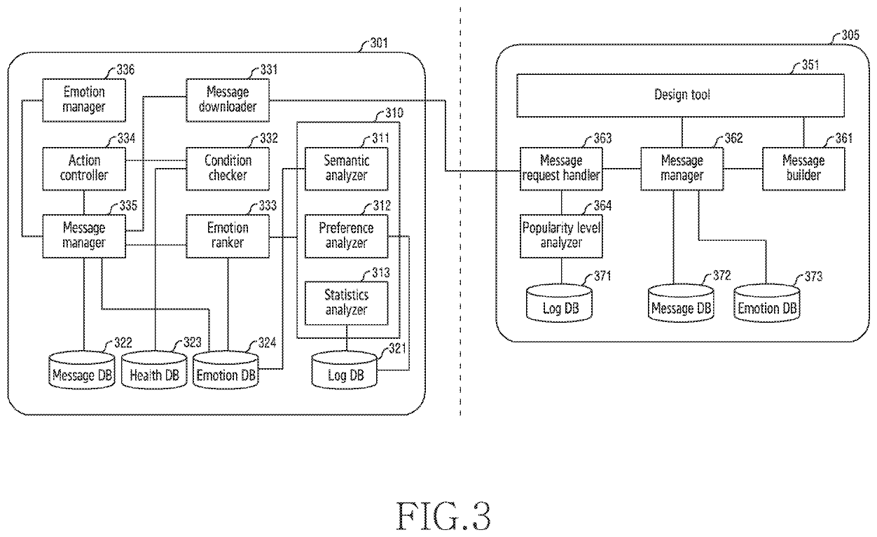

According to an embodiment, the memory 240 may at least temporarily store various types of information used for providing coaching to a user. For example, the memory 240 may store at least some of user profile information (e.g., an ID, a password, a biometric ID, a log-in state, a log-in history, an age, a gender, a height, a weight, or an illness) associated with a user of the electronic device 200, user biometric data, health information (e.g., sleep information, exercise information, diet information and/or illness information) obtained by processing the user biometric data, a health information analysis result (e.g., a sleep analysis result, an excise evaluation result, a diet management result, and/or an illness-related monitoring result), or various databases (e.g., a log database 321, a message database 322, a health database 323, an emotion database 324 of FIG. 3).

According to an embodiment, the sensor module 250 may include at least one sensor. For example, the sensor module 250 may include one or more of an acceleration sensor, a gyro sensor, a motion sensor, a biometric sensor (e.g., a photoplethysmogram (PPG) sensor, an electrocardiography (ECG) sensor, a galvanic skin response (GSR) sensor, a bioelectrical impedance analysis (BIA) sensor, a blood glucose sensor, a blood pressure sensor, or a body fat sensor).

For example, the sensor module 250 may output user movement data, user biometric data and/or health information obtained by processing the biometric data (e.g., sleep information, excise information, diet information and/or illness information). The biometric data that the sensor module 250 outputs may include, for example, at least some data among data obtained by performing pre-processing such as reducing noise from sensed raw data and/or data obtained by performing post-processing such as matching to a previously stored pattern. According to an embodiment, the electronic device 200 may provide user movement data via a motion sensor. The motion sensor detects at least one of a user exercise state (e.g., walking, running), a sleep state (e.g., a state of being unused due to sleep, tossing and turning), and an emergency state (e.g., collapse). The electronic device 200 may detect user biometric data (e.g., blood oxygen saturation, a heart rate, a blood glucose, a blood pressure, a body fat, a sleep state, an exercise state, a diet state, biometric data during sleep, biometric data during exercise, or biometric data during having a meal) via a biometric sensor. The user movement data and/or health information processed using biometric data may be provided.

In addition, the type of sensor included in the sensor module 250 is not limited. For example, the sensor module 250 may further include various sensors such as a distance sensor (e.g., an ultrasonic sensor, an optical sensor, a time of flight (ToF)), and an olfactory sensor, and may use the same for a coaching function. For example, coaching associated with a good posture for measurement may be provided in order to measure biometric information. In addition, although not illustrated, the electronic device 200 may include a camera module (e.g., the camera module 180 of FIG. 1), and may use the same for a coaching function. For example, a user's diet may be captured or a user's skin condition may be measured using the camera module 150.

According to an embodiment, the communication circuit 230 may include a wireless communication module (e.g., the wireless communication module 192 of FIG. 1) ((e.g., a cellular communication module, a short-range wireless communication module, or a global navigation satellite system (GNSS) communication module)).

According to an embodiment, the communication circuit 230 may support the connection of short-range wireless communication to the electronic device 200. For example, the communication circuit 230 may support the connection of short-range wireless communication (e.g., Bluetooth, Bluetooth low energy (LE), wireless fidelity (WiFi) direct, or infrared data association (IrDA)) between the electronic device 200 and an external electronic device (e.g., a smartphone carried while a user exercises, a smartphone located in a short distance when a user sleeps, a weighing machine, a medical device, and/or a wearable device that a user is wearing).

The electronic device 200 may obtain user health information via the sensor module 250, or may obtain user health information via an external electronic device (e.g., a wearable device such as a smart watch) connected in short-range wireless communication.

According to an embodiment, the communication circuit 230 may support the connection of long-range wireless communication to the electronic device 200. For example, the communication circuit 230 may receive information associated with a healthcare service and/or a coaching service from an external electronic device 305 in the long-range wireless communication.

According to an embodiment, the communication circuit 230 may provide location information including a global navigation satellite system (GNSS). The electronic device 200 may receive current location information (place information such as a home or office, a gym, or a restaurant) using a GNSS, and may use the same for a coaching function. For example, in the case that the location of the electronic device 200 is detected as a gym, the electronic device 200 may provide coaching related to exercise to a user. Alternatively, in the case that the location of the electronic device 200 is detected as a restaurant, the electronic device 200 may provide coaching related to a diet to a user.

According to an embodiment, the processor 210 may provide a user interface for coaching. A user interface for coaching may be provided in various forms.

For example, the user interface for coaching may include a visual type of user interface. The user interface for coaching may be embodied as a hybrid-type of user interface including two or more of a visual type of user interface, an auditive type of user interface (e.g., audio type, sound type), and a tactile type of user interface (e.g., vibration).

The electronic device 200 may include an output module (e.g., at least one of the display 220, the sound module 260, or the haptic module 270) for providing a user interface.

The processor 210 may provide (or display) a visual type of user interface via the display 220. The processor 210 may provide (or output) an auditive type of user interface via the sound module 260. The processor 210 may provide (or output) a tactile type of user interface via the haptic module 270.

According to an embodiment, the processor 210 of the electronic device 200 may detect the occurrence of a coaching event. The electronic device 200 may perform a coaching function in response to the detection of the occurrence of the coaching event. The coaching function may be provided for managing and/or improving the user's health condition. According to an embodiment, the coaching function may be embodied as at least one instruction or at least one application module. For example, the coaching function is a function included in a health application, and may be included in the health application as at least one instruction. In this instance, the case in which an instruction related to the coaching function (e.g., an instruction related to determining the content of coaching, an instruction for outputting the determined content of coaching) is performed by the processor 210 in the state in which the health application is executed may be defined as the case in which the coaching function is performed. According to another embodiment, the case in which a coaching function is loaded as a separate application or application module, in a memory (e.g., the volatile memory 132 of FIG. 1), and is performed by the processor 210 may be defined as the case in which a coaching function is performed.

For example, in the case that a result obtained by analyzing user health information (e.g., a sleep analysis result, an exercise evaluation result, a diet management result, and/or an illness-related monitoring result) satisfies a designated condition, the processor 210 may detect the occurrence of a coaching event.

As another example, when a designated function (e.g., updating today's sleep score) is performed by a predetermined application (e.g., a health application), the processor 210 may detect the occurrence of a coaching event.

As another example, in the case that a device context satisfies a designated condition (e.g., when an alarm time arrives, when a display is turned on in the state in which a coaching function is turned on), or in the case that a user input for requesting coaching is present (e.g., touching a coaching button), the processor 210 may detect the occurrence of a coaching event.

As another example, in the case that a predetermined application (e.g., a health application) is performed in the electronic device 200, in the case that a predetermined object (e.g., a button, a menu) is selected on an application execution screen that is being displayed on the screen of the electronic device 200, or in the case that a coaching request is received from an external electronic device (e.g., a smart watch) connected to short-range wireless communication to the electronic device 200 (e.g., Bluetooth, Wi-Fi), the processor 210 may detect the occurrence of a coaching event.

When a coaching event occurs, a coaching function may be triggered. For example, the case in which a coaching function is triggered may include an operation in which a coaching function starts its performance.

According to an embodiment, the processor 210 may determine a coaching message to be displayed based on a coaching event. For example, a coaching event occurs in association with a user health information analysis result, the processor 210 may determine a coaching message (or an original coaching message or coaching content) to be displayed based on the analysis result.

For example, a coaching message may include the content of coaching to be provided to a user (e.g., at least some of a title, a core content, a detailed description, and miscellanies. The content of coaching may include text, but it is not limited thereto. For example, the content of coaching may include an object obtained by imaging text. As another example, the content of coaching may include one or more of an emoticon, an object, an icon, an image, or a graphic element that is to express a content corresponding to text, is to be added to text, or is to be displayed together with text. The content of coaching may be stored in the memory 240 for each element. For example, some of the content of coaching may be omitted and output according to the level of details of a user interface that is set by a user. For example, in the case in which a user sets, using a setting menu, the level of details for a user interface including only a title, a core content, and an emoticon, respective elements are mapped to each other and stored in the form of a data table in the memory 240 so that the electronic device 200 is capable of selecting only a title, a core content, and an emoticon among the content of coaching.

According to an embodiment, the processor 210 may identify at least one emotion tag related to a coaching message.

According to an embodiment, at least one emotion tag related to a coaching message may include a representative emotion tag (e.g., rapture) and one or more associated-emotion tags (e.g., being touched, admired, moved, happy, or hopeful). For example, at least one emotion tag related to a coaching message may be emotion tag(s) same as or similar to the emotion tag of the coaching message. The emotion tag of the coaching message may be an emotion tag included in the coaching message. The emotion tag of the coaching message may correspond to a representative emotion tag. The representative emotion tag may be the emotion tag having the strongest association with the content of coaching among the emotion tags included in an emotion information model stored in advance.

According to an embodiment, the processor 210 may determine, based on user context information, a representative visual element in a visual element candidate group corresponding to at least one emotion tag.

According to an embodiment, the processor 210 may display a visual type of user interface including a coaching message and a representative visual element via the display 220.

For example, each visual element included in a visual element candidate group may include at least one of an emoticon, an object, an icon, an image, a graphic element, a moving emoticon, a moving picture, or an animation element.

According to an embodiment, a visual element candidate group may include a plurality of visual elements.

Based on at least one emotion tag related to a coaching message, the electronic device 200 may select a visual element candidate group that includes a number of visual elements, wherein the number may be up to a designated threshold value (e.g., 10).

In the case that, when selecting a visual element candidate group is performed, the number of visual elements capable of being candidates is greater than the designated threshold value, only a representative emotion tag may be taken into consideration. Conversely, in the case that the number of visual elements capable of being candidates is less than the threshold value, a secondary associated-emotion tag may be taken into consideration, in addition to a primary associated-emotion tag.

According to various embodiments, the electronic device 200 may determine a coaching message to be provided to a user according to a user health information analysis result (e.g., a sleep analysis result, an exercise evaluation result, a diet management result, and/or illness-related monitoring result), may identify an emotion tag related to the coaching message, and may provide a visual element (e.g., a visual element included in the visual element candidate group and/or a representative visual element) associated with the coaching message using the emotion tag.

According to various embodiments, an emotion tag related to a coaching message may be an emotion tag corresponding to an expected user emotion associated with the coaching message, and the electronic device 200 may provide a visual element using the emotion tag. However, the range of the embodiments is not limited thereto. For example, the electronic device 200 may use a second emotion tag indicating emotion information associated with the condition of health by replacing a first emotion tag corresponding to an expected user emotion with the second emotion tag, or may use the second emotion tag in addition to the first emotion tag. The electronic device 200 may provide a visual element related to emotion information associated with a health condition (e.g., an illness-related monitoring score) to a user using the second emotion tag. The second emotion tag may be emotion information associated with a user biometric signal state and/or a user illness-related state. For example, the second emotion tag may express emotion information associated with a condition such as a high blood glucose, a low blood glucose, a high blood pressure, a low blood pressure, the abnormality of a heart rate pattern, but the disclosure is not limited thereto. For example, criterion information (e.g., a data table associated with emotions mapped for each monitoring score or according to a change of a monitoring score) for identifying emotion information according to a health condition (e.g., an illness-related monitoring score) may be stored in advance.

Although various embodiments disclosed in the document illustrate that the electronic device 200 is a device of the type of smartphone, the type of electronic device is not limited thereto, and may be embodied as various types such as a smartphone, a flexible smartphone, a wearable device (e.g., a smart watch, smart glasses), or a tablet.

The configuration of the electronic device 200 illustrated in FIG. 2 is merely an example, but does not limit the range of embodiments, and may be modified, expanded, and/or applied in various forms.

According to an embodiment, the electronic device 200 may include all of the sensor module 250 for collecting data, and the display 220, the sound module 260, and the haptic module 270 that are output modules for providing a user interface.

For example, the processor 210 of the electronic device 200 may output a user interface for coaching via an output module (e.g., at least one of the display 220, the sound module 260, or the haptic module 270). The processor 210 may output a visual type of user interface, an auditive type of user interface, a tactile type of user interface, or a hybrid type of user interface to a user via an output module.

According to an embodiment, the electronic device 200 (e.g., one of a smartphone and a wearable device of a user) may interoperate with an external electronic device (e.g., the other one between the smartphone and the wearable device of the user), and may use a module (e.g., a sensor module, a display, a haptic module, and a sound module) of the external electronic device for coaching.

For example, the electronic device 200 may be in the state of being connected to an external electronic device in the short-range wireless communication. The electronic device 200 may provide a user interface for coaching using an output module that the electronic device itself is equipped with and/or an output module of the external electronic device. For example, the processor 210 of the electronic device 200 may transmit information associated with a user interface to the external electronic device via the communication circuit 230 so that the external electronic device is capable of outputting the user interface (e.g., a screen, text, sound, vibration). For example, in the state in which the electronic device 200 (e.g., a smartphone), which a user carries while doing an exercise, sleeping, or having a meal, is connected to an external electronic device (e.g., a smart watch), which a user is wearing, in the short-range wireless communication, the electronic device 200 may transmit information associated with a user interface for coaching so as to output the user interface via the smart watch.

According to an embodiment, the electronic device 200 may provide coaching using both a module that the electronic device itself is equipped with and/or a module of an external electronic device. For example, the electronic device 200 may collect different types of biometric data from the sensor module 250 that the electronic device itself is equipped with and a sensor module of the external electronic device.

Although not illustrated, in an embodiment, the electronic device 200 may further include an input device (e.g., a touch sensor of the display module 160 of FIG. 1 or the camera module 180), and may collect data (e.g., diet data) usable for coaching using the same.

In an embodiment, a user interface to be provided for coaching may be provided differently according to a device context (e.g., whether the display 220 of the electronic device 200 is turned on/off, whether an external electronic device is present that is being connected to the electronic device 200 in the short-range wireless communication, or whether a user wears the electronic device 200 or the external electronic device) at the point in time at which a coaching event occurs.

For example, the electronic device 200 may identify device context information when a coaching event occurs. In the case that the result of the identification shows that the user is using the electronic device 200 (e.g., when the display 220 of the electronic device 200 is turned on), a user interface for coaching may be output via the output module (e.g., at least one of the display 220, the sound module 260, and haptic module 270) of the electronic device 200. In the case that the identification result shows that the electronic device 200 (e.g., the smartphone of a user) is not being used, and the user is wearing an external electronic device (e.g., a smart watch of the user) (e.g., when it is detected that the display 220 of the electronic device 200 is in the turned-off state and the user is in the state of wearing the sensor module of the external electronic device), a user interface for coaching may be output via the output module of the external electronic device.

According to an embodiment, the electronic device 200 may perform synchronization with at least one external electronic device (e.g., a smart watch) and/or a server (e.g., the server 108 of FIG. 1) via the communication circuit 230. For example, the electronic device 200 may synchronize at least some of sensing data, health information, and/or whether a coaching function is used (e.g., whether the content of coaching is provided, whether a user checks out the content of coaching). Through the above, although there is a history of disconnection of the connection to an external electronic device and/or a server, or a history of powered-off of the electronic device 200 or the external electronic device, an experience of using a consecutive coaching function may be provided to the user.

FIG. 3 is a block diagram illustrating the configuration of each module of an electronic device and an external electronic device according to an embodiment.

According to various embodiments, the electronic device 301 may include an additional component element in addition to the component elements illustrated in FIG. 3, or may omit at least one of the component elements illustrated in FIG. 3. Each component element illustrated in FIG. 3 may not be necessarily embodied as hardware which is physically distinguished. For example, each component element illustrated in FIG. 3 may be a software element.

According to an embodiment, the electronic device 301 of FIG. 3 may correspond to the electronic device 101 of FIG. 1 or the electronic device 200 of FIG. 2. The external electronic device 305 illustrated in FIG. 3 may correspond to the server 108 of FIG. 1, or may correspond to a service server that supports a healthcare service and/or a coaching service.

According to an embodiment, a processor (e.g., the processor 210 of FIG. 2) of an electronic device (e.g., the electronic device 200 of FIG. 2) to embody the component elements illustrated in FIG. 3 may implement instructions stored in a memory (e.g., the memory 240 of FIG. 2), and may control hardware (e.g., the communication circuit 230, the display 220, the sound module 260, or the haptic module 270) associated with an operation and/or function.

Referring to FIG. 3, the electronic device 301 according to an embodiment may include an emotion analyzer 310, a log database 321, a message database 322, a health database 323, an emotion database 323, a message download 331, a condition checker 332, an emotion ranker 333, an action controller 334, a message manager 335, and an emotion manager 336.

The emotion analyzer 310 may analyze user health information and/or user context information in response to a request from the emotion ranker 333, and may return an analysis result to the emotion ranker 333. The analysis result may include one or more emotion tags related to a coaching message and/or scoring information associated with a visual element candidate group capable of being included in the coaching message.

The emotion analyzer 310 may include a semantic analyzer 311, a preference analyzer 312, and a statistics analyzer 313.

The semantic analyzer 311 may select a visual element candidate group using an emotion information model stored in advance in the emotion database 324. Each visual element included in the visual element candidate group may be mapped to an emotion tag that is the same as or similar to an emotion tag of the coaching message. The emotion tag of the coaching message may be an emotion tag included in the coaching message. The emotion tag of the coaching message may correspond to a representative emotion tag.

The semantic analyzer 311 may make an analysis based on the semantic similarity between a coaching message and a visual element, and may select, based on an analysis result, the visual element candidate group for the coaching message.

The semantic analyzer 311 may extract, based on the emotion information model stored in advance, a plurality of emotion tags that are the same as or similar to the emotion tag of the coaching message to be provided to a user, and may select visual elements mapped to the extracted emotion tags as the visual element candidate group.

For example, the emotion information model may be provided in a tree structure configured with a plurality levels of nodes (or branches). The emotion information model may include a plurality of levels of nodes, a pair of highest-level nodes (e.g., negative emotion, positive emotion), high-level nodes branched out from each highest-level node (e.g., delight, pride, love, fear, anger, compassion, shame, despair, grief), and low-level nodes branched out from each high-level node.

For example, in the case that the emotion tag of the coaching message is ‘rapture’ and determining emotion tags related to the coaching message is performed using the tree-structured emotion information model stored in advance, a representative emotion tag is ‘rapture’, primary associated-emotion tags may be emotion tags (e.g., being touched, admired, moved) presenting in the same node as that of the ‘rapture’, and secondary associated-emotion tags may be emotion tags (e.g., happy or hope) having the same parent node as that of the ‘rapture’. The semantic analyzer 311 may include visual elements mapped to the corresponding emotion tags (e.g., rapture, touched, admired, moved, happy, and hope) in a temporary visual element candidate group. Subsequently, the semantic analyzer 311 may select visual elements to be finally included in the visual element candidate group in order of the ‘representative emotion tag>a primary associated-emotion tag>a secondary associated-emotion tag’. In this instance, a threshold value (a maximum of 10 elements) for the scale of the visual element candidate group may be designated. For example, in the case that a visual element candidate group that satisfies the designated threshold value is determined based on the representative emotion tag, the primary or secondary associated-emotion tag may not be taken into consideration.

In addition, various types of semantic analysis may be performed via the semantic analyzer 311. For example, the semantic analyzer 311 may make a morphological analysis of the text that is one of the component elements of the coaching message, and may automatically extract an emotion tag.

The preference analyzer 312 may analyze, based on log information stored in the log database 321, user's preference for the visual elements in the visual element candidate group. For example, in association with each visual element and/or a coaching message including the visual elements, the preference analyzer 312 may analyze user preference for each visual element in consideration of at least one among the period of time during which the coaching message is preserved in the electronic device 200 (the difference between the exposure time and deletion time of the coaching message), the number of times that user interaction is performed (e.g., the number of times that a video provided as a visual element is clicked (or reproduced)), whether the detailed content of the coaching message is identified, and a user feedback associated with a visual element (e.g., an input to an object in the coaching message (e.g., whether a like/dislike button is selected, whether a button for identifying the detailed content is clicked)). The preference analyzer 312 may return a user preference score for each visual element in the visual element candidate group.

The statistics analyzer 313 may analyze, based on log information stored in the log database 321, the statistics of usage of the visual elements in the visual element candidate group. For example, the statistics analyzer 313 may analyze the latest usage history associated with each visual element (e.g., the latest time at which each visual element is shown to a user and/or the number of times that each visual element is shown to a user during a designated recent period (e.g., N days)). The statistics analyzer 313 may return an exposure statistics score for each visual element in the visual element candidate group.

The message downloader 331 may download an application (e.g., a health application, an exercise application, a fitness application, a sleep application, and a diet management application) for a healthcare service and/or a coaching service from the external electronic device 305 in response to a user request. In an application execution environment, the message downloader 331 may download message information associated with coaching messages provided from the external electronic device 305, and may store the same in the message database 322 via the message manager 335. The message downloader 331 may update the message database 322 periodically or aperiodically. For example, the message downloader 331 may update the message database 322 via a server (e.g., the server 108 of FIG. 1). Alternatively, the message database 322 may be updated by the message downloader 331 internally in the electronic device 200. For example, at least part of message information may be updated based on a user feedback (e.g., whether the content of coaching is satisfied, the frequency of identification), or when at least part of another database (e.g., a user profile, health information, the health DB 323, the log DB 321) is updated, this may be monitored and updating may be performed.

The condition checker 332 may obtain user health information (e.g., at least some of sleep information, exercise information, diet information, and illness information) and may store the same in the health database 323.

The condition checker 332 may analyze the user health information, and may transfer an analysis result (e.g., a sleep analysis result, an exercise evaluation result, a diet management result, and/or an illness-related monitoring result) to the action controller 334. For example, the condition checker 332 may compare the current condition based on the user health information and a previously set goal condition, and may provide a comparison result.

The action controller 334 may detect the occurrence of a coaching event. For example, the action controller 334 may receive the analysis result obtained by analyzing the user health information from the condition checker 332, and when the analysis result satisfies a designated condition, the action controller 334 may determine that a coaching event occurs.

When a coaching event occurs, the action controller 334 may transfer, to the message manager 335, event information (e.g., an event identifier, an event type, an event occurrence time point, the content of an event, a device context (e.g., whether a display is turned on/off, a battery state) at the point in time at which an event occurs) associated with the coaching event.

The message manager 335 may determine a coaching message to be displayed based on the event information received from the action controller 334. For example, the message management 335 may extract, from the message database 322, the coaching message to be displayed according to the event information received among a plurality of coaching messages stored in advance.

The emotion manager 336 may receive a new visual element group (e.g., third party emoticons) from the external electronic device 305 via an application programming interface (API). The external electronic device 305 may provide, based on a predetermined data protocol, additional information (e.g., the frequency of use and preference associated with each visual element) associated with the new visual element group together.

The emotion ranker 333 may receive a coaching message to be displayed from the message manager 335, and may identify one or more emotion tags related to the coaching message using the emotion information model stored in the emotion database 324.

In addition, based on visual element information stored in the emotion database 324, the emotion ranker 333 may select a visual element candidate group capable of being included in the coaching message (or a visual element candidate group corresponding to the one or more emotion tags).

The emotion ranker 333 may rank (ranking) the visual elements included in the visual element candidate group. The emotion ranker 333 may request analyzing the visual element candidate group for ranking, and may receive an analysis result returned. The analysis result may include scoring information (e.g., a user preference score, an exposure statistics score) associated with each visual element in the visual element candidate group. The emotion ranker 333 may perform scoring that applies a weight to each visual element in the visual element candidate group using the scoring information, and may determine the priorities of the visual elements.

The emotion ranker 333 may select a representative visual element in the visual element candidate group according to the priority, i.e., weighted score, of each visual element. The representative visual element may be included in the coaching message and be provided to a user.

The log database 321 may store log information. The log information may include user context information. For example, user context information may include information associated with at least one of a user feedback, usage statistics, user preference, and a popularity level for each of a plurality of visual elements. For example, in association with each visual element and/or a coaching message including the visual elements, the user context information may store information associated with at least one among the period of time during which the coaching message is preserved in the electronic device 301 (the difference between the exposure time and deletion time of the coaching message), the number of times that user interaction is performed (e.g., the number of times that a video provided as a visual element is clicked (or reproduced)), whether the detailed content of the coaching message is identified, user feedback associated with a visual element (e.g., an input to an object in the coaching message (e.g., whether a like/dislike button is selected, whether a button for identifying the detailed content is clicked)), and the latest usage history (e.g., the latest time at which each visual element is shown to a user and/or the number of times that each visual element is shown to a user during a designated recent period (e.g., N days)). The emotion ranker 333 may use the information stored in the log database 321 to calculate one or more of the user preference score and the exposure statistics score.

The message database 322 may store message information associated with coaching messages. In addition, event information associated with event conditions for exposing each coaching message may be stored as associated information of message information. In the case that an event designated by event information occurs, the message manager 335 may extract a coaching message to be displayed from the message database 322 in response to the event, and may transfer the same to the emotion ranker 333.

The health database 323 may store health information of a user. For example, the health information may include at least some of sleep information (e.g., sleeping hours), exercise information (e.g., the number of steps, the duration of exercise), and diet information (e.g., mealtime, caloric intake). In addition, as information associated with health information, condition information for coaching conditions may be stored. The condition checker 332 may determine that a coaching event occurs when a condition designated by the condition information is satisfied.

The emotion database 324 may store an emotion information model. The emotion information model may include tag information associated with a plurality of emotion tags. The plurality of emotion tags included in the emotion information model may be defined in advance. In addition, the emotion database 324 may store visual element information for at least one visual element mapped to each emotion tag of the emotion information model. The emotion information model, tag information, and/or visual element information may be updated or distributed from the external electronic device 305 at regular periods.

Referring to FIG. 3, the external electronic device 305 may include a design tool 351, a message builder 361, a message manager 362, a message request handler 363, a popularity level analyzer 364, a log database 371, a message database 372, and an emotion database 373.

The message database 372 may store overall information (e.g., application information, service information, or message information) that the external electronic device 305 manages for supporting a healthcare service and/or coaching service. The external electronic device 305 may provide, to the electronic device 301, message information associated with a plurality of coaching messages stored in the message database 372 in the case that a request from the electronic device 301 is present.

The emotion database 373 may store an emotion information model. The emotion information model may include tag information associated with a plurality of emotion tags. The plurality of emotion tags included in the emotion information model may be defined in advance. In addition, the emotion database 373 may store visual element information associated with at least one visual element mapped to each emotion tag of the emotion information model. The external electronic device 305 may update the electronic device 301 with the emotion information model, the tag information and/or the visual element information stored in the emotion database 373, or may distribute the same to the electronic device 301 at regular periods.

The log database 371 may store log information related to a healthcare service and/or a coaching service. For example, the log information may include at least one among user profile information (e.g., login information for each user (e.g., an ID, a password, a biometric ID, a login state, and a login history) associated with a plurality of users, physical information for each user (e.g., an age, a gender, a height, a weight), health information for each user, coaching history information for each user, and evaluation criterion information (e.g., statistics information, popularity level information, preference information) for the entire visual elements usable for coaching. The log database 371 may be updated periodically or aperiodically via a connection to the external electronic device 305 or a server (e.g., the server 108 of FIG. 1) using a user input to the electronic device 301 and/or a communication circuit (e.g., the communication circuit 230 of FIG. 2). For example, the log database 371 may be updated by interoperating with the server (e.g., the server 108 of FIG. 1) related to a healthcare service and/or a coaching service. For example, the electronic device 301 may transmit the user's age information and/or gender information to the server 108, and the server 108 may receive group information (e.g., age group information, gender group information) determined based thereon and may update the log database 371.

The design tool 351 may correspond to a development tool for supporting a service. For example, an application (e.g., a health application, an exercise application, or a diet management application) including a coaching function, or coaching messages used for the coaching function may be produced, verified, distributed, and/or updated using the design tool 351.

The message request handler 363 may process a request from the electronic device 301. The message request handler 363 may provide, to the electronic device 301, message information associated with coaching messages stored in the message database 372 in response to a request from the electronic device 301.

The message builder 361 may interpret an input via the design tool 351, may configure a coaching message based on the input, and may transfer the same to the message manager 362.

The message manager 362 may provide an interface that is capable of reading and writing coaching messages. The message manager 362 may store coaching messages configured by the design tool 351 or the message builder 361 in the message database 372, and in the case that a request from the message request handler 363 is present, a coaching message may be extracted from the message database 372 and may be provided in response to the corresponding request.

The popularity level analyzer 364 may analyze log information stored in the log database 371, may recognize the popularity level based on a user profile (e.g., an age group, a gender) of the electronic device 301, and may provide the corresponding popularity level information to the electronic device 301.

The configuration of the electronic device 301 and/or the external electronic device 305 illustrated in FIG. 3 is merely an example, but does not limit the range of embodiments, and may be modified, expanded, and/or applied in various forms.

For example, the electronic device 301 and/or external electronic device 305 may include only some of the illustrated component elements or may further include other component elements.

The structure of the database may be embodied in a form different from the example of FIG. 3. For example, the log databases 321 and 371, the message databases 322 and 372, the health database 323, and the emotion databases 324 and 373 may be combined or separated in a different manner from that of FIG. 3. Databases may be embodied independently, or in the form in which at least some of databases are integrated. At least some of the databases are integrated, and one of the electronic device 301 and the external electronic device 305 may store the integrated database and share the same with the other.

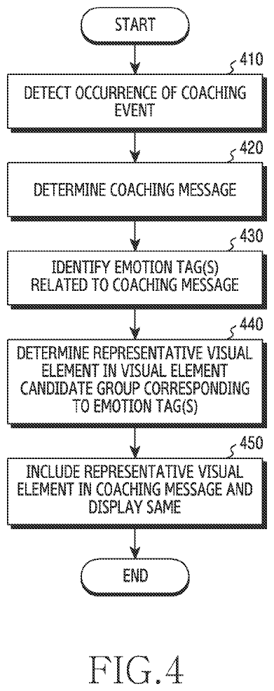

FIG. 4 is a flowchart illustrating an operation method of an electronic device according to an embodiment.

For example, the method illustrated in FIG. 4 may correspond to the operation method of an electronic device for providing coaching. The method of FIG. 4 may be performed by an electronic device (e.g., the electronic device 200 of FIG. 2, the processor 210, or an application (e.g., a health application) executed in the electronic device 200). For ease of description, it is assumed that the operation method of FIG. 4 is performed by the processor 210 of the electronic device 200, but the disclosure is not limited thereto.

Referring to FIG. 4, an operation method of an electronic device according to an embodiment may include operation 410, operation 420, operation 430, operation 440, and operation 450. Operations of FIG. 4 may be performed sequentially, parallelly, repeatedly, or heuristically, or one or more operations may be performed in a different order or may be omitted, or one or more operations may be added.

In operation 410, the processor 210 of the electronic device 200 may detect the occurrence of a coaching event.

For example, in the case that a result obtained by analyzing user health information (e.g., a sleep analysis result, an exercise evaluation result, a diet management result, an illness-related monitoring result) satisfies a designated condition, the electronic device 200 may detect the occurrence of a coaching event. In order to detect the occurrence of a coaching event, condition information associated with coaching conditions may be stored in advance in the memory 240 (e.g., the health database 323) in the electronic device 200.

As another example, when a designated function (e.g., updating today's sleep score) is performed by a predetermined application (e.g., a health application), the electronic device 200 may detect the occurrence of a coaching event.

As another example, in the case that a device context satisfies a designated condition (e.g., when an alarm time arrives, when a display is turned on in the state in which a coaching function is turned on), or in the case that a user input for requesting coaching is present (e.g., touching a button for triggering a coaching function), the electronic device 200 may detect the occurrence of a coaching event.

As another example, in the case that a predetermined application (e.g., a health application) is performed in the electronic device 200, in the case that a predetermined object (e.g., a button, a menu) is selected on an application execution screen that is being displayed on the screen of the electronic device 200, or in the case that a coaching request is received from an external electronic device (e.g., a smart watch) connected to the electronic device 200 in the short-range wireless communication (e.g., Bluetooth, Wi-Fi), the electronic device 200 may detect the occurrence of a coaching event.

In operation 420, the processor 210 of the electronic device 200 may determine a coaching message to be displayed based on the coaching event.

In one embodiment, a coaching message associated with the coaching event that occurs, message information associated with coaching messages and/or event information associated with event conditions may be stored in advance in the memory 240 (e.g., the message database 322) in the electronic device 200.

For example, the coaching message may include the content of coaching to be provided to a user (e.g., at least some of a title, a core content, a detailed description, and miscellanies). The content of coaching may include text, but it is not limited thereto. For example, the content of coaching may include an object obtained by imaging text. As another example, the content of coaching may include one or more from among an emoticon, an object, an icon, an image, or a graphic element that is to express a content corresponding to text, is to be added to text, or is to be displayed together with text.

In operation 430, the processor 210 of the electronic device 200 may identify at least one emotion tag related to the coaching message determined in operation 420.

According to an embodiment, at least one emotion tag related to the coaching message may include a representative emotion tag (e.g., rapture) and one or more associated-emotion tags (e.g., being touched, admired, moved, happy, hopeful). For example, the at least one emotion tag related to the coaching message may be the emotion tag(s) same as or similar to the emotion tag of the coaching message. The emotion tag of the coaching message may be an emotion tag included in the coaching message. The emotion tag of the coaching message may correspond to a representative emotion tag. The representative emotion tag may be the emotion tag having the strongest association with the content of coaching among the emotion tags included in an emotion information model stored in advance.

For example, the electronic device 200 may identify one or more emotion tags related to the coaching message based on the emotion information model stored in advance.

In operation 440, the processor 210 of the electronic device 200 may determine, based on user context information, a representative visual element in a visual element candidate group corresponding to at least one emotion tag.

For example, each visual element included in the visual element candidate group may include at least one of an emoticon, an object, an icon, an image, a graphic element, a moving emoticon, a moving picture, or an animation element.

According to an embodiment, a visual element candidate group may include a plurality of visual elements.

Based on at least one emotion tag related to the coaching message, the electronic device 200 may select a visual element candidate group so as to include multiple visual elements in the coaching message. In one embodiment, the number of visual elements is a designated threshold value (e.g., 10).

In the case that, when selecting a visual element candidate group is performed, the number of visual elements capable of being candidates is greater than the designated threshold value, only a representative emotion tag may be taken into consideration. Conversely, in the case that the number of visual elements capable of being candidates is less than the threshold value, a secondary associated-emotion tag may be taken into consideration, in addition to a primary associated-emotion tag.

For example, the electronic device 200 may extract a representative emotion tag from the coaching message. In the case that the number of visual elements mapped to the representative emotion tag is greater than or equal to a threshold value, the electronic device 200 may select as many visual element candidates to be included in a candidate group as the threshold value from the visual elements mapped to the representative emotion tag. In the case that the number of the visual elements mapped to the representative emotion tag is less than the threshold value, the electronic device 200 may identify a primary associated-emotion tag of the representative emotion tag. In the case that the number of visual elements mapped to the representative emotion tag and the primary associated-emotion tag is greater than or equal to the threshold value, the electronic device 200 may select as many visual element candidates as threshold value from the visual elements mapped to the representative emotion tag and the primary associated-emotion tag.

In the case that the number of the visual elements mapped to the representative emotion tag and the primary associated-emotion tag is less than the threshold value, the electronic device 200 may identify a secondary associated-emotion tag of the representative emotion tag. The electronic device 200 may select as many visual element candidates as the threshold value from the visual elements mapped to the representative emotion tag, the primary associated-emotion tag, and the secondary associated-emotion tag.

According to an embodiment, the processor 210 of the electronic device 200 may select a visual element candidate group of the coaching message via semantic analysis that analyzes the semantic similarity between the coaching message and the visual element. The processor 210 may evaluate, based on log information stored in the memory 240, preference for each visual element in the visual element candidate group. The processor 210 may evaluate, based on the log information stored in the memory 240, non-preference for each visual element in the visual element candidate group. Based on a preference evaluation result and a non-preference evaluation result, the processor 210 may adjust the number of visual elements included in the visual element candidate group to the threshold value (e.g., 10).

The electronic device 200 may select, using user context information, a representative visual element among a plurality of visual elements included in the visual element candidate group.

According to an embodiment, user context information that is a criterion for selecting the representative visual element may include information associated with at least one of a user feedback, usage statistics, user preference, and a popularity level for each of the plurality of visual elements. These criterion may be embodied in one or more of a user preference score and the exposure statistics score.

According to an embodiment, the representative visual element to be included in the coaching message may adaptively vary based on the user context information. For example, based on the current user context information, the representative visual element may be selected from the visual element candidate group. For example, when the user context information is updated, the representative visual element may be dynamically determined, based on the updated user context information, in the visual element candidate group.

The representative visual element may be an element for intuitively and understandably expressing the coaching message determined in operation 420 (or an original coaching message or the content of coaching), for assigning emotion to coaching, or for improving fun or unexpectedness of coaching.

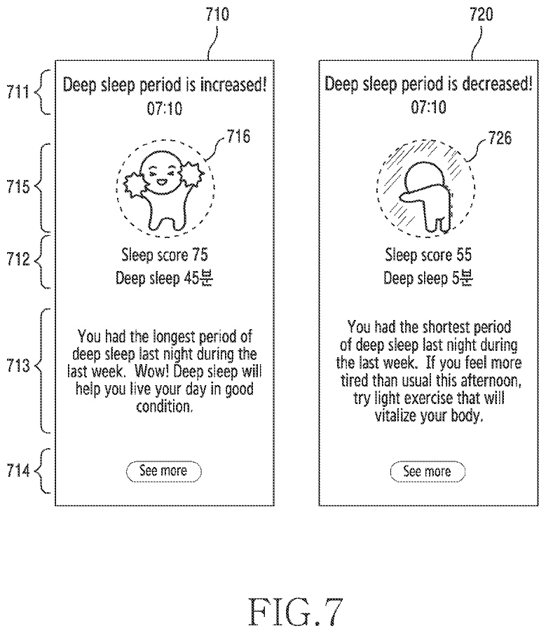

In operation 450, the processor 210 of the electronic device 200 may include the representative visual element determined in operation 440 in the coaching message, and may display the same via the display 220. The coaching message displayed via the display 220 may include the content of coaching and the representative visual element. A visual type of user interface (e.g., any one of a first screen 710 of a second screen 720 of FIG. 7 and a first screen 810, a second screen 820, a third screen 830, or a fourth screen 840) including the coaching message may be provided (or displayed).

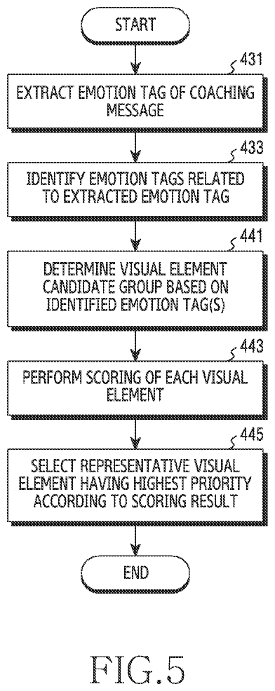

FIG. 5 is a flowchart illustrating part of the operation method of the electronic device of FIG. 4.

For example, the operation 430 of FIG. 4 may include operation 431 and operation 433 of FIG. 5. The operation 440 of FIG. 4 may include operation 441 and operation 443 of FIG. 5.

In operation 431, the electronic device 200 may extract an emotion tag (e.g., congratulation) of a coaching message to be displayed. The emotion tag (e.g., congratulation) of the coaching message extracted in operation 431 may correspond to a representative emotion tag.

For example, the coaching message may include a tag identifier, or may be mapped to a tag identifier and stored. The electronic device 200 may extract the emotion tag of the coaching message via the tag identifier included in the coaching message or mapped to the coaching message. As another example, the electronic device 200 may extract the emotion tag of the coaching message by making a morphological analysis of text included in the coaching message.

In an embodiment, in the case that two or more emotion tags are included in the coaching message, the electronic device 200 may select one of the emotion tags as the emotion tag of the coaching message. For example, based on an emotion information model stored in advance, an emotion tag in the highest node among the emotion tags in the coaching message, or an emotion tag that most frequently appears in the coaching message may be selected as the emotion tag of the coaching message.

In operation 433, the electronic device 200 may identify one or more emotion tags (e.g., festivity, self-congratulation) related to the emotion tag extracted from the coaching message. The one or more emotion tags identified via operation 433 may correspond to associated-emotion tags. For example, the electronic device 200 may discover associated-emotion tags (e.g., festivity, self-congratulation) of the representative emotion tag (e.g., congratulation) using the emotion information model stored in advance.

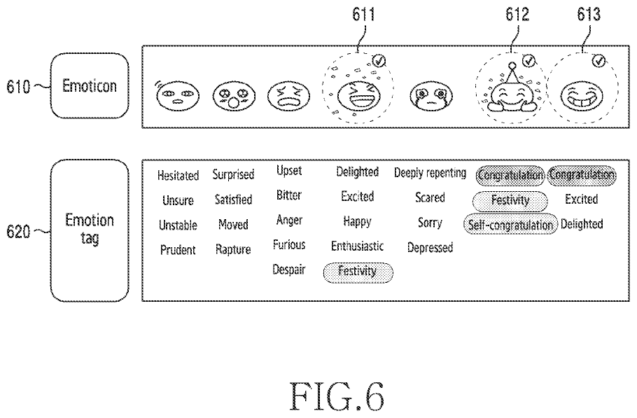

In operation 441, the electronic device 200 may determine a visual element candidate group based on the emotion tag (or the representative emotion tag, e.g., congratulation) of the coaching message extracted in operation 431 and one or more emotion tags (e.g., associated-emotion tags, e.g., festivity and self-congratulation) identified via operation 433. The visual element candidate group may include a plurality of visual elements mapped to the plurality of emotion tags (e.g., congratulation, festivity, and self-congratulation). An example of the mapping relationship between a visual element and an emotion tag that is a criterion for configuring a visual element candidate group is illustrated in FIG. 6.

In operation 443, the electronic device 200 may perform scoring with respect to each visual element included in the visual element candidate group. In operation 445, the electronic device 200 may determine, based on a scoring result obtained in operation 443, a visual element having the highest priority in the visual element candidate group as a representative visual element.

According to an embodiment, to determine the representative visual element, the electronic device 200 may perform, based on user context information, scoring with respect to each visual element in the visual element candidate group.

The user context information that is a criterion associated with scoring may include information associated with at least one of a user feedback, usage statistics, user preference, and a popularity level for each of a plurality of visual elements. Here, some (e.g., usage statistics) of the user context information may be a negative scoring element that lowers a scoring mark. For example, in the visual element candidate group, a low weight may be allocated to a visual element that has a high frequency of appearance in the usage statistics during a predetermined period (e.g., 7 days). Other some (e.g., user feedback, user preference, and a popularity level) of the user context information may be a positive scoring element that increases a scoring mark. For example, in the visual element candidate group, a high weight may be allocated to a visual element having a high accumulated user feedback score, a visual element having a high accumulated user preference score, or a visual element having a high popularity level for users in the same age group.

The electronic device 200 may perform, based on the user context information, scoring with respect to visual elements that belong to the visual element candidate group, and may select, based on a scoring result, a visual element having the highest priority as the representative visual element.

The emotion information model usable in the electronic device according to an embodiment will be described as follows.

According to an embodiment, the coaching message to be displayed may be related to one or more emotion tags. For example, in association with one or more emotion tags related to the coaching message, various emotion tags may be identified using a tree-structure emotion information model defined and/or classified as a plurality of levels of categories (or nodes or branches). The electronic device 200 may extract emotion tags that are the same as or similar to the emotion tag of the coaching message from the emotion information model.

The emotion tags included in the tree-structured emotion information model may be classified as a positive emotion category and a negative emotion category. In a lower level of each emotion category, a plurality of detailed emotion tags may be included.

The coaching message may be related to a plurality of emotion tags.

For example, in the case that the emotion tag (or the representative emotion tag) of the coaching message is ‘rapture’, emotion tags of ‘being touched, admired, moved, rapture’ presenting in the same node (or branch) as that of the ‘rapture’ in the emotion information model, and emotion tags of ‘happy or hope’ having the same parent node as that of the ‘rapture’ and presenting closest to the corresponding node may be identified as emotion tags related to the coaching message. The visual elements mapped to the identified emotion tags may be included in the visual element candidate group and may be considered as candidates of the representative visual element.

FIG. 6 is a diagram illustrating an example of the mapping relationship between visual elements and emotion tags in order to describe a scheme of determining a representative visual element by an electronic device according to an embodiment.

According to an embodiment, a visual element may include an emoticon. The electronic device 200 may identify at least one emotion tag related to a coaching message and an emoticon candidate group corresponding to the at least one emotion tag.

In the example of FIG. 6, diagram 610 corresponds to a plurality of emoticons. Diagram 620 corresponds to a plurality of emotion tags. As illustrated, one or more emotion tags may be mapped to each emoticon.

For example, an emotion tag (or a representative emotion tag) of a coaching message to be displayed is ‘congratulation’, and associated-emotion tags may be ‘festivity’ and ‘self-congratulation’. The emotion information for each emoticon is tagged as illustrated in FIG. 6, a first emoticon 611, a second emoticon 612, and a third emoticon 613 mapped to the corresponding emotion tags (congratulation, festivity, and self-congratulation) may be included in an emoticon candidate group.

In the case that, when selecting a visual element candidate group is performed, the number of visual elements capable of being candidates is greater than a designated threshold value, only a representative emotion tag may be taken into consideration. Conversely, in the case that the number of visual elements capable of being candidates is less than the threshold value, a secondary associated-emotion tag may be taken into consideration, in addition to a primary associated-emotion tag.

FIG. 7 is a diagram illustrating examples of user interfaces displayable in an electronic device according to an embodiment.

The electronic device 200 may display a visual type of user interface such as a first screen 710 or a second screen 720. The first screen 710 or the second screen 720 illustrates the configuration of a user interface including a coaching message. The coaching message may include the content of coaching and a representative emoticon associated with the content of coaching.

The first screen 710 is the case in which a coaching message including a first emoticon 716 is displayed. The second screen 720 is the case in which a coaching message including a second emoticon 726 is displayed.

A user interface including a coaching message may include a coaching content display areas 711, 712, 713, and 714 and an emoticon display area 715. The content of coaching may be displayed in the coaching content display areas 711, 712, and 713. For example, the content of coaching may include at least some of the title in display area 711, the core content in display area 712, the detailed descriptions in display area 713, and miscellanies.

The coaching content display areas may include a function area 714. The function area 714 may display an object (e.g., a button, a menu) for providing a designated function (e.g., see more of the coaching content) related to the content of coaching.

The first emoticon 716 that is a representative emoticon related to the content of coaching may be displayed in the emoticon display area 715.

For example, in the case in which the content of coaching is related to emotion tags (e.g., congratulation, festivity, and self-congratulation) corresponding to a positive emotion, the electronic device 200 may select, as the representative emoticon, the first emoticon 716 among the emoticon candidates corresponding to the emotion tags, and may display the same via the emoticon display area 715.

As another example, in the case in which the content of coaching is related to emotion tags (e.g., getting upset, depressed, and despairing) corresponding to a negative emotion, the electronic device 200 may select, as the representative emoticon, the second emoticon 726 among the emoticon candidates corresponding to the emotion tags, and may display the same together with the corresponding content of coaching.

According to an embodiment, a user interface including a coaching message may be embodied variously according to settings. For example, the electronic device 200 may determine, based on a user input, the level of details of a user interface that provides the content of coaching. For example, the level of details may show which item among the content of coaching is to be included in the user interface. For example, in the case of the highest level of details, the user interface may include all of the content of coaching (e.g., the title 711, the core content 712, the detailed descriptions 713, and the first emoticon 716). As another example, in the case of the lowest details, the user interface may include only the first emoticon 716. The details of the user interface may be determined by user settings and/or by the electronic device 200 according to settings, and the configuration of the user interface is not limited.

According to an embodiment, although FIG. 7 illustrates that a single coaching content is included in the entirety of the first screen 710, the disclosure is not limited thereto. For example, each of the various coaching contents (e.g., a coaching content related to exercise and a coaching content related to a diet) may be included as an interface in the form of a card in the user interface.

According to an embodiment, by providing an emoticon together as one of the elements of the coaching message, a coaching content may be provided to a user in an intuitive and understandable manner. In addition, by analyzing user health information, discovering user-customized coaching content, expressing the coaching content best, and dynamically determining an emoticon that empathizes with the situation of a user, a user's interest in the coaching message may be improved, and a user may feel as if the user always would receive a new guidance. Although a message including the same or similar content may be repeatedly provided from the perspective of the characteristic of coaching, such method may enable a user feel less bored. In addition, such method may be implemented so as to respond to (or to reproduce) a predetermined user motion such as touching an emoticon, and may provide fun and unexpectedness. Accordingly, the retention of a coaching service that may be stodgy and boring may be increased and the effect of coaching may be also increased.

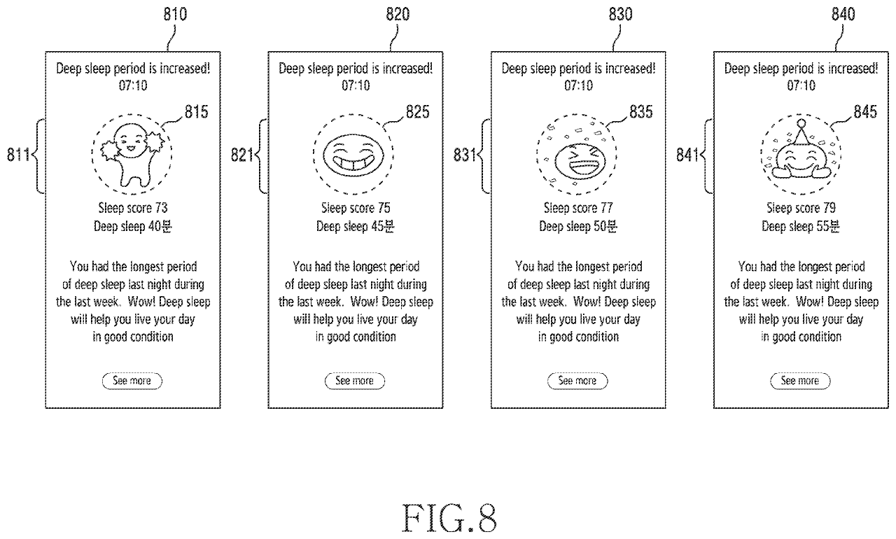

FIG. 8 is a diagram illustrating other examples of user interfaces displayed in an electronic device according to an embodiment.

According to an embodiment, although the content of coaching of coaching messages are the same as, or similar to each other, different emoticons may be selected for the contents of coaching.

A first screen 810, a second screen 820, a third screen 830, and a fourth screen 840 of FIG. 8 illustrate the case of including different emoticons (e.g., a first emoticon 815, a second emoticon 825, a third emoticon 835, and a fourth emoticon 845) in the same or similar coaching messages, and displaying the same.

For example, in the case that the sleep analysis result shows that today's sleep score falls within a designated range (e.g., in the range of 70 to 80 points), and the deep sleeping hours are 40 minutes to 60 minutes, the content of coaching may be determined as ‘You had the longest period of deep sleep last night during the last week. Wow! Deep sleep will help you live your day in good condition.’

An emoticon candidate group related to the corresponding content of coaching may include the first emoticon 815, the second emoticon 825, the third emoticon 835, and the fourth emoticon 845.

The electronic device 200 may select a representative emoticon among the emoticon candidate group by taking into consideration of user context information (e.g., a user feedback, usage statistics, user preference, and the level of popularity).

For example, the electronic device 200 may perform, based on the user context information, scoring with respect to emoticons that belong to the emoticon candidate group, and may select a representative emoticon having the highest priority according to a scoring result. In one embodiment, the scoring includes calculating a one or more of a user preference score and an exposure statistics score.

An emoticon to be displayed together with the coaching content may adaptively vary according to the user context information (e.g., user feedback, usage statistics, user preference, and the level of popularity). Based on the current user context, a representative emoticon may be selected from among the emoticon candidate group. For example, when user context information is updated periodically or in response to an event (an update event, analysis event), a representative emoticon may be dynamically determined, based on the updated user context information, from the emoticon candidate group.

The first screen 810 may be an example of the case in which the first emoticon 815 is selected as a representative emoticon from the emoticon candidate group, and is displayed. The first emoticon 815 may be displayed via an emoticon display area 811 of the first screen 810.

The second screen 820 may be an example of the case in which the second emoticon 825 is selected as a representative emoticon from the emoticon candidate group, and is displayed. The second emoticon 825 may be displayed via an emoticon display area 821 of the second screen 820.

The third screen 830 may be an example of the case in which the third emoticon 835 is selected as a representative emoticon from the emoticon candidate group, and is displayed. The third emoticon 835 may be displayed via an emoticon display area 831 of the third screen 830.

The fourth screen 840 may be an example of the case in which the fourth emoticon 845 is selected as a representative emoticon from the emoticon candidate group, and is displayed. The fourth emoticon 845 may be displayed via an emoticon display area 841 of the fourth screen 840.

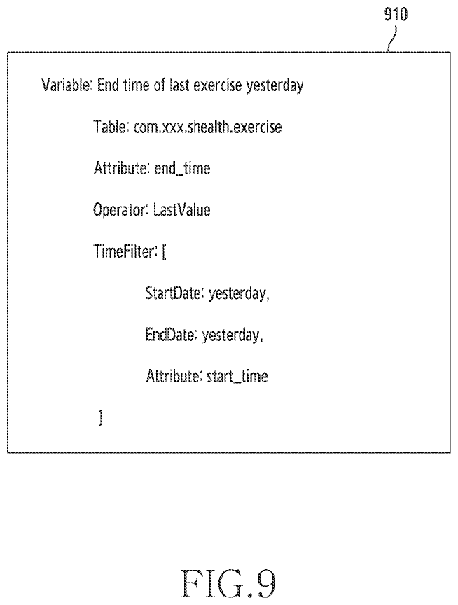

FIG. 9 is a diagram illustrating an example to describe a coaching condition of an electronic device according to an embodiment.

Table 1 below illustrates condition information associated with coaching conditions.

TABLE 1

| | |
|---|---|
| Condition | today's sleep score is lower than the average score of a |
| example 1 | user's age group |
| | Variable: today's sleep score |
| | Operator: Less than |
| | Value: Average sleep score of user's age group |
| Condition | The end time of last exercise of yesterday is later than |
| example 2 | [bedtime − 3 H] |
| | Variable: End time of last exercises yesterday |
| | Operator: later than |
| | Variable: Bedtime − 3 H |
| Condition | The dinner intake calories of yesterday are greater than |
| example 3 | 1/3 of a target value |
| | Variable: Dinner intake calories yesterday |
| | Operator: Greater than |
| | Variable: Target value X 1/3 |

In Table 1, a variable may be an analysis result value obtained by analyzing user health information. For example, in the condition example 2, a variable (end time of last exercise yesterday) may be a result value obtained by reading all exercise records of yesterday from a table storing exercise information, and returning a record having the latest exercise end time.

FIG. 9 is a diagram illustrating how a variable is described in detail.

Diagram 910 may be a variable type that defines a coaching condition. Referring to FIG. 9, it is recognized that the corresponding variable (end time of last exercise yesterday) is related to exercise information (e.g., exercise records) among user health information.

An operator may be an operator for comparing a variable and a value, and for comparing a variable and a variable, and for comparing a value and a value. A value may be a constant value.

Condition information including a set of the above-described conditions may be stored. When a designated condition based on condition information is satisfied, a coaching message corresponding to the corresponding condition may be provided to a user.

The electronic device 200 may determine whether a designated condition is satisfied based on the condition information set in advance (e.g., whether today's sleep score is lower than the average score of a user's age group, whether the end time of the last exercise of yesterday is later than a time corresponding to 3 hours before sleep, whether the caloric intake at the dinner of the last night is greater than ⅓ of a caloric goal), and may detect the occurrence of a coaching event based on the determination. In the case that the designated condition is satisfied, the electronic device 200 may display a coaching message for a corresponding coaching event.

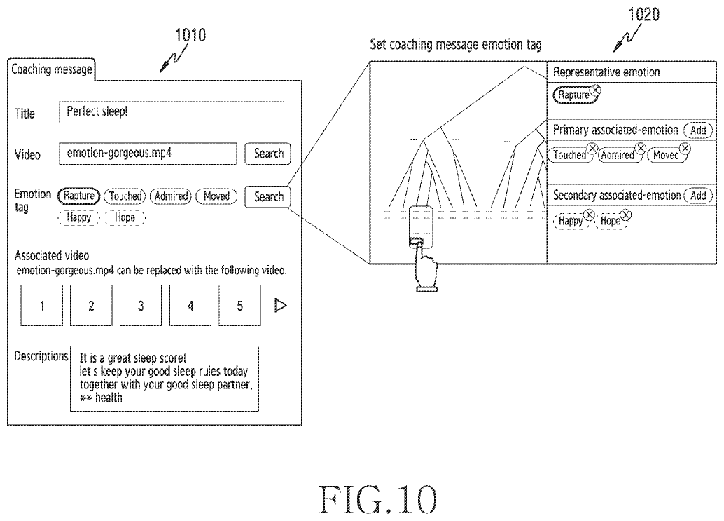

FIG. 10 is a diagram illustrating an example of a scheme of setting a coaching message and an emotion tag using a design tool according to an embodiment.

A design tool of FIG. 10 may correspond to the design tool 351 of the external electronic device 305 illustrated in FIG. 3. A user may set the coaching content of a coaching message and/or an emotion tag mapped to the coaching content using the design tool.

In the example of FIG. 10, diagram 1010 may be a coaching message setting screen. Diagram 1020 may be an emotion tag setting screen of a coaching message.

Emotion tags related to the coaching message may include a single representative emotion tag (e.g., rapture) and a plurality of associated-emotion tags (e.g., being touched, admired, moved, happy, or hopeful). The representative emotion tag and emotion tags that are present in the same node or a sibling node having the same parents, that is, primary associated-emotion tags (e.g., being touched, admired, moved) and secondary associated-emotion tags (e.g., happy, hopeful) may be automatically set as emotion tags related to the coaching message.

The automatically set emotion tags may appear in the coaching message setting screen 1010.

The emotion tag setting screen 1020 may appear according to a user input (e.g., touching a discover button) to the coaching message setting screen 1010. The emotion tags automatically set by the emotion tag setting screen 1020 as emotion tags related to the coaching message may be added or deleted by a user.

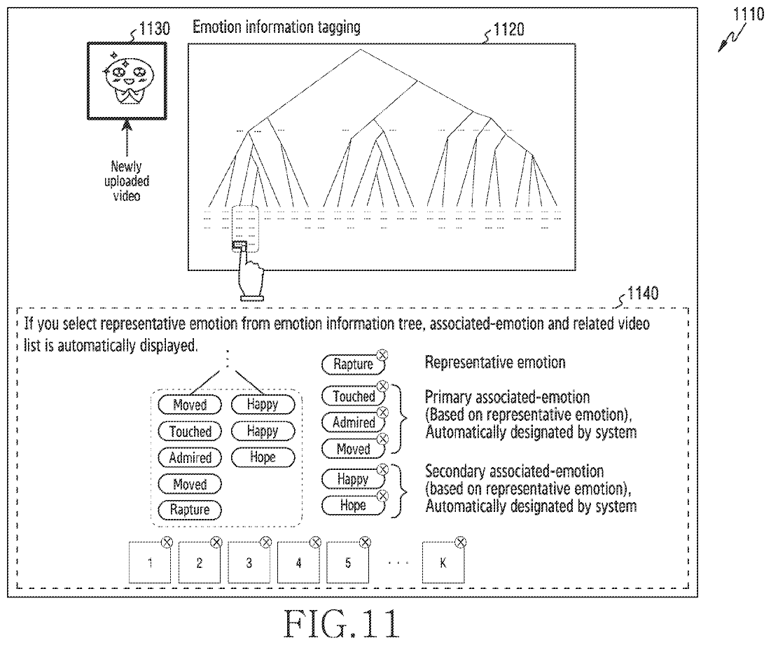

FIG. 11 is a diagram illustrating an example of a scheme that registers a new visual element using a design tool according to an embodiment.

A design tool of FIG. 11 may correspond to the design tool 351 of the external electronic device 305 illustrated in FIG. 3. A user may register a new visual element using the design tool. Information associated with the new visual element may be stored locally in the electronic device 301 (e.g., the emotion database 324 of the electronic device 301), or may be uploaded to an external electronic device 305 (e.g., the emotion database 373 of the external electronic device 305).

The diagram 1110 may be a visual element registration screen. For example, the visual element registration screen 1110 may include a first area 1120, a second area 1130, and a third area 1140, as illustrated in the drawing. An emotion information model may be displayed in the first area 1120. A new visual element to be registered may be displayed in the second area 1130. Tag information associated with emotion tags to be mapped to the new visual element may be displayed in the third area 1140. According to a user input to the emotion information model of the second area 1130, an emotion tag to be mapped to the new visual element may be added or deleted.

The electronic device according to various embodiments may be one of various types of electronic devices. The electronic devices may include, for example, a portable communication device (e.g., a smartphone), a computer device, a portable multimedia device, a portable medical device, a camera, a wearable device, or a home appliance. According to an embodiment of the disclosure, the electronic devices are not limited to those described above.

It should be appreciated that various embodiments of the present disclosure and the terms used therein are not intended to limit the technological features set forth herein to particular embodiments and include various changes, equivalents, or replacements for a corresponding embodiment. With regard to the description of the drawings, similar reference numerals may be used to refer to similar or related elements. It is to be understood that a singular form of a noun corresponding to an item may include one or more of the things, unless the relevant context clearly indicates otherwise. As used herein, each of such phrases as “A or B,” “at least one of A and B,” “at least one of A or B,” “A, B, or C,” “at least one of A, B, and C,” and “at least one of A, B, or C,” may include any one of, or all possible combinations of the items enumerated together in a corresponding one of the phrases. As used herein, such terms as “1st” and “2nd,” or “first” and “second” may be used to simply distinguish a corresponding component from another, and does not limit the components in other aspect (e.g., importance or order). It is to be understood that if an element (e.g., a first element) is referred to, with or without the term “operatively” or “communicatively”, as “coupled with,” “coupled to,” “connected with,” or “connected to” another element (e.g., a second element), it means that the element may be coupled with the other element directly (e.g., wiredly), wirelessly, or via a third element.

As used in connection with various embodiments of the disclosure, the term “module” may include a unit implemented in hardware, software, or firmware, and may interchangeably be used with other terms, for example, “logic,” “logic block,” “part,” or “circuitry”. A module may be a single integral component, or a minimum unit or part thereof, adapted to perform one or more functions. For example, according to an embodiment, the module may be implemented in a form of an application-specific integrated circuit (ASIC).

Various embodiments as set forth herein may be implemented as software (e.g., the program #40) including one or more instructions that are stored in a storage medium (e.g., internal memory #36 or external memory #38) that is readable by a machine (e.g., the electronic device #01). For example, a processor (e.g., the processor #20) of the machine (e.g., the electronic device #01) may invoke at least one of the one or more instructions stored in the storage medium, and execute it, with or without using one or more other components under the control of the processor. This allows the machine to be operated to perform at least one function according to the at least one instruction invoked. The one or more instructions may include a code generated by a complier or a code executable by an interpreter. The machine-readable storage medium may be provided in the form of a non-transitory storage medium. Wherein, the term “non-transitory” simply means that the storage medium is a tangible device, and does not include a signal (e.g., an electromagnetic wave), but this term does not differentiate between where data is semi-permanently stored in the storage medium and where the data is temporarily stored in the storage medium.

According to an embodiment, a method according to various embodiments of the disclosure may be included and provided in a computer program product. The computer program product may be traded as a product between a seller and a buyer. The computer program product may be distributed in the form of a machine-readable storage medium (e.g., compact disc read only memory (CD-ROM)), or be distributed (e.g., downloaded or uploaded) online via an application store (e.g., PlayStore™), or between two user devices (e.g., smart phones) directly. If distributed online, at least part of the computer program product may be temporarily generated or at least temporarily stored in the machine-readable storage medium, such as memory of the manufacturer's server, a server of the application store, or a relay server.

According to various embodiments, each component (e.g., a module or a program) of the above-described components may include a single entity or multiple entities, and some of the multiple entities may be separately disposed in different components. According to various embodiments, one or more of the above-described components may be omitted, or one or more other components may be added. Alternatively or additionally, a plurality of components (e.g., modules or programs) may be integrated into a single component. In such a case, according to various embodiments, the integrated component may still perform one or more functions of each of the plurality of components in the same or similar manner as they are performed by a corresponding one of the plurality of components before the integration. According to various embodiments, operations performed by the module, the program, or another component may be carried out sequentially, in parallel, repeatedly, or heuristically, or one or more of the operations may be executed in a different order or omitted, or one or more other operations may be added.

An electronic device (e.g., one of the electronic device 200 of FIG. 2 or the electronic device 301 of FIG. 3) according to various embodiments may include a memory (e.g., the memory 240 of FIG. 2), a display (e.g., the display 220 of FIG. 2), a communication circuit (e.g., the communication circuit 230 of FIG. 2), and at least one processor (e.g., the processor 210 of FIG. 2). The at least one processor may be operatively connected to the memory, the display, and the communication circuit. The memory may store instructions that, when executed, cause the at least one processor to detect occurrence of a coaching event, to determine a coaching message to be displayed based on the coaching event, to identify at least one emotion tag related to the coaching message, to determine, based on user context information of a user, a representative visual element in a visual element candidate group corresponding to the at least one emotion tag, and to include the representative visual element in the coaching message and display the same via the display.

According to various embodiments, the at least one emotion tag may include a representative emotion tag and one or more associated-emotion tags.

According to various embodiments, the instructions, when executed, may cause the at least one processor to select, based on the at least one emotion tag, the visual element candidate group including visual elements of which a number corresponds to a threshold value.

According to various embodiments, the instructions, when executed, may cause the at least one processor to extract a representative emotion tag from the coaching message, to select the visual element candidate group from visual elements mapped to the representative emotion tag in the case that the number of visual elements mapped to the representative emotion tag is greater than a threshold value, to identify a primary associated-emotion tag of the representative emotion tag in the case that the number of visual elements mapped to the representative emotion tag is less than the threshold value, and to select the visual element candidate group from visual elements mapped to the representative emotion tag and the primary associated-emotion tag in the case that the number of visual elements mapped to the representative emotion tag and the primary associated-emotion tag is greater than or equal to the threshold value.

According to various embodiments, the instructions, when executed, may cause the at least one processor to identify a secondary associated-emotion tag of the representative emotion tag in the case that the number of visual elements mapped to the representative emotion tag and the primary associated-emotion tag is less than the threshold value, and to select the visual element candidate group from visual elements mapped to the representative emotion tag, the primary associated-emotion tag, and the secondary associated-emotion tag.

According to various embodiments, when the user context information is updated, a representative visual element may be dynamically determined, based on the updated user context information, from the visual element candidate group.

According to various embodiments, the user context information may include information associated with at least one of a user feedback, usage statistics, user preference, and a popularity level associated with each of the plurality of visual elements.

According to various embodiments, the instructions, when executed, may cause the at least one processor to perform scoring of each visual element in the visual element candidate group based on the user context information in order to determine the representative visual element. Here, part of the user context information is a negative scoring element that lowers a scoring mark, and the other part of the user context information is a positive scoring element that increases a scoring mark.

According to various embodiments, the instructions, when executed, may cause the at least one processor to select a visual element candidate group of a coaching message via a semantic analysis that analyzes semantic similarity between the coaching message and a visual element, to evaluate, based on log information stored in the memory, preference for each visual element in the visual element candidate group, to evaluate, based on the log information, non-preference for each visual element in the visual element candidate group, and to adjust, based on a preference evaluation result and a non-preference evaluation result, the number of visual elements included in the visual element candidate group to a threshold value.

According to various embodiments, the electronic device may further include one or more from among a sound module and a haptic module. The instructions, when executed, may cause the at least one processor to output, via the sound module, an auditive type of user interface corresponding to the representative visual element, or to output, via the haptic module, a tactile type of user interface corresponding to the representative visual element.

An operation method of an electronic device according to various embodiments may include an operation of detecting occurrence of a coaching event, an operation of determining a coaching message to be displayed based on the coaching event, an operation of identifying at least one emotion tag related to the coaching message, an operation of determining, based on user context information of a user, a representative visual element in a visual element candidate group corresponding to the at least one emotion tag, and an operation of including the representative visual element in the coaching message and displaying the same on a display of the electronic device.

According to various embodiments, the at least one emotion tag may include a representative emotion tag and one or more associated-emotion tags.

According to various embodiments, the method may further include an operation of selecting, based on the at least one emotion tag, the visual element candidate group including visual elements of which a number is a threshold value.

According to various embodiments, the operation of selecting the visual element candidate group may include an operation of extracting a representative emotion tag from the coaching message, an operation of selecting the visual element candidate group from one or more visual elements mapped to the representative emotion tag in the case that the number of visual elements mapped to the representative emotion tag is greater than or equal to a threshold value, an operation of identifying a primary associated-emotion tag of the representative emotion tag in the case that the number of visual elements mapped to the representative emotion tag is less than the threshold value, and an operation of selecting the visual element candidate group from one or more visual elements mapped to the representative emotion tag and the primary associated-emotion tag in the case that the number of visual elements mapped to the representative emotion tag and the primary associated-emotion tag is greater than or equal to the threshold value.

According to various embodiments, the operation of selecting the visual element candidate group may include an operation of identifying a secondary associated-emotion tag of the representative emotion tag in the case that the number of visual elements mapped to the representative emotion tag and the primary associated-emotion tag is less than the threshold value, and an operation of selecting the visual element candidate group from visual elements mapped to the representative emotion tag, the primary associated-emotion tag, and the second associated-emotion tag.

According to various embodiments, when the user context information is updated, a representative visual element may be dynamically determined, based on the updated user context information, in the visual element candidate group.

According to various embodiments, the user context information may include information associated with at least one of a user feedback, usage statistics, user preference, and a popularity level associated with each of a plurality of visual elements.

According to various embodiments, the operation of determining the representative visual element may include an operation of performing scoring of each visual element in the visual element candidate group based on the user context information. Here, part of the user context information may be a negative scoring element that lowers a scoring mark and the other part of the user context information may be a positive scoring element that increases a scoring mark.

According to various embodiments, the operation of selecting the visual element candidate group may include an operation of selecting the visual element candidate group of the coaching message via a semantic analysis that analyzes semantic similarity between the coaching message and a visual element, an operation of evaluating, based on log information stored in the electronic device, preference for each visual element in the visual element candidate group, an operation of evaluating, based on the log information, non-preference for each visual element in the visual element candidate group, and an operation of adjusting, based a preference evaluation result and a non-preference evaluation result, the number of visual elements included in the visual element candidate group to a threshold value.

According to various embodiments, the method may further include an operation of outputting, via a sound module of the electronic device, an auditive type of user interface corresponding to the representative visual element, and an operation of outputting, via a haptic module, a tactile type of user interface corresponding to the representative visual element.

1. An electronic device comprising:
a memory;
a display;
a communication circuit; and
at least one processor operatively connected to the memory, the display, and the communication circuit,
wherein the memory stores instructions that, when executed, cause the at least one processor to:
detect occurrence of a coaching event;
determine a coaching message to be displayed based at least in part on the coaching event;
identify at least one emotion tag related to the coaching message;
determine, based at least in part on user context information of a user, a representative visual element from a visual element candidate group corresponding to the at least one emotion tag; and
include the representative visual element in the coaching message and display the coaching message via the display.

2. The electronic device of claim 1, wherein the at least one emotion tag comprises a representative emotion tag and one or more associated-emotion tags.

3. The electronic device of claim 1, wherein the instructions, when executed, cause the at least one processor to select, based at least in part on the at least one emotion tag, the visual element candidate group including a number of visual elements.

4. The electronic device of claim 3, wherein the instructions, when executed, cause the at least one processor to:
extract a representative emotion tag from the coaching message;
based at least in part on a determination that the number of the visual elements mapped to the representative emotion tag is greater than a threshold value, select the visual element candidate group from visual elements mapped to the representative emotion tag;
based at least in part on a determination that the number of the visual elements mapped to the representative emotion tag is less than the threshold value, identify a primary associated-emotion tag of the representative emotion tag, and
based at least in part on a determination that the number of the visual elements mapped to the representative emotion tag and the primary associated-emotion tag is greater than the threshold value, select the visual element candidate group from visual elements mapped to the representative emotion tag and the primary associated-emotion tag.

5. The electronic device of claim 4, wherein the instructions, when executed, cause the at least one processor to:
based at least in part on a determination that the number of the visual elements mapped to the representative emotion tag and the primary associated-emotion tag is less than the threshold value, identify a secondary associated-emotion tag of the representative emotion tag; and
select the visual element candidate group from visual elements mapped to the representative emotion tag, the primary associated-emotion tag, and the secondary associated-emotion tag.

6. The electronic device of claim 1, wherein, when the user context information is updated, a representative visual element is dynamically determined, based at least in part on the updated user context information, in the visual element candidate group.

7. The electronic device of claim 1, wherein the user context information comprises information associated with at least one of a user feedback, usage statistics, user preference, and a popularity level associated with each of the plurality of visual elements.

8. The electronic device of claim 1, wherein the instructions, when executed, cause the at least one processor to perform scoring of each visual element in the visual element candidate group based at least in part on the user context information in order to determine the representative visual element, and
part of the user context information is a negative scoring element that lowers a scoring mark, and the other part of the user context information is a positive scoring element that increases a scoring mark.

9. The electronic device of claim 1, wherein the instructions, when executed, cause the at least one processor to:
select a visual element candidate group of a coaching message via a semantic analysis that analyzes semantic similarity between the coaching message and a visual element;
evaluate, based at least in part on log information stored in the memory, preference for each visual element in the visual element candidate group;
evaluate, based at least in part on the log information, non-preference for each visual element in the visual element candidate group; and
adjust, based at least in part on a preference evaluation result and a non-preference evaluation result, a number of visual elements included in the visual element candidate group to a threshold value.

10. The electronic device of claim 1, further comprising one or more from among a sound module and a haptic module, and
wherein the instructions, when executed, cause the at least one processor to output, via the sound module, an auditive type of user interface corresponding to the representative visual element, or to output, via the haptic module, a tactile type of user interface corresponding to the representative visual element.

11. An operation method of an electronic device for providing coaching, the method comprising:
detecting occurrence of a coaching event;
determining a coaching message to be displayed based at least in part on the coaching event;
identifying at least one emotion tag related to the coaching message;
determining, based at least in part on user context information of a user, a representative visual element in a visual element candidate group corresponding to the at least one emotion tag; and
including the representative visual element in the coaching message and displaying the coaching message on a display of the electronic device.

12. The method of claim 11, wherein the at least one emotion tag comprises a representative emotion tag and one or more associated-emotion tags.

13. The method of claim 11, further comprising selecting, based at least in part on the at least one emotion tag, the visual element candidate group including a number of visual elements.

14. The method of claim 13, wherein the selecting of the visual element candidate group comprises:
extracting a representative emotion tag from the coaching message;
based at least in part on a determination that a number of the visual elements mapped to the representative emotion tag is greater than or equal to a threshold, selecting the visual element candidate group from one or more visual elements mapped to the representative emotion tag value;
based at least in part on a determination that the number of visual elements mapped to the representative emotion tag is less than the threshold value, identifying a primary associated-emotion tag of the representative emotion tag; and
based at least in part on a determination that a number of the visual elements mapped to the representative emotion tag and the primary associated-emotion tag is greater than or equal to the threshold value selecting the visual element candidate group from one or more visual elements mapped to the representative emotion tag and the primary associated-emotion tag.

15. The method of claim 14, wherein the selecting of the visual element candidate group comprises:
based at least in part on a determination that the number of the visual elements mapped to the representative emotion tag and the primary associated-emotion tag is less than the threshold value, identifying a secondary associated-emotion tag of the representative emotion tag and
selecting the visual element candidate group from visual elements mapped to the representative emotion tag, the primary associated-emotion tag, and the second associated-emotion tag.

16. The method of claim 11, wherein, when the user context information is updated, a representative visual element is dynamically determined, based at least in part on the updated user context information, in the visual element candidate group.

17. The method of claim 11, wherein the user context information comprises information associated with at least one of a user feedback, usage statistics, user preference, and a popularity level associated with each of a plurality of visual elements.

18. The method of claim 11, wherein the determining of the representative visual element comprises performing scoring of each visual element in the visual element candidate group based at least in part on the user context information, and
part of the user context information is a negative scoring element that lowers a scoring mark and the other part of the user context information is a positive scoring element that increases a scoring mark.

19. The method of claim 13, wherein the selecting of the visual element candidate group comprises:
selecting the visual element candidate group of the coaching message via a semantic analysis that analyzes semantic similarity between the coaching message and a visual element;
evaluating, based at least in part on log information stored in the electronic device, preference for each visual element in the visual element candidate group;
evaluating, based at least in part on the log information, non-preference for each visual element in the visual element candidate group; and
adjusting, based a preference evaluation result and a non-preference evaluation result, a number of visual elements included in the visual element candidate group to a threshold value.

20. The method of claim 19, further comprising:
outputting, via a sound module of the electronic device, an auditive type of user interface corresponding to the representative visual element; and
outputting, via a haptic module of the electronic device, a tactile type of user interface corresponding to the representative visual element.

21. The electronic device of claim 1, wherein the communication circuit is in communication with an external electronic device that is being worn by the user, and wherein the instructions, when executed, further cause the at least one processor to:
detect whether the user is interacting with the electronic device; and
based at least in part on a determination that the user is not interacting with the electronic device, transmit the coaching message to the external electronic device.

22. The electronic device of claim 1, wherein identifying the at least one emotion tag related to the coaching message comprises performing a morphological analysis of a text included in the coaching message.

23. The electronic device of claim 1, wherein determining the representative visual element from the visual element candidate group includes calculating at least one of a user preference score and an exposure statistics score.
